(19) United States
(12) Patent Application Publication
(10) Pub. No.: US 2014/0148423 A1
(43) Pub. Date: May 29, 2014

(54) PHARMACEUTICAL COMPOSITION COMPRISING A TRPA1 ANTAGONIST AND A STEROID

(71) Applicants: Neelima Khairatakar-Joshi, Thane (W) (IN); Abhay Kulkarni, Navi Mumbai (IN); Dinesh Pradeep Wale, Osmanabad (IN); Anil Hari Kadam, Ahmednagar (IN); Vikram Bhosale, Mumbai (IN)

(72) Inventors: Neelima Khairatakar-Joshi, Thane (W) (IN); Abhay Kulkarni, Navi Mumbai (IN); Dinesh Pradeep Wale, Osmanabad (IN); Anil Hari Kadam, Ahmednagar (IN); Vikram Bhosale, Mumbai (IN)

(73) Assignee: GLENMARK PHARMACEUTICALS S.A., La Chaux-de-Fonds (CH)

(21) Appl. No.: 14/233,300

(22) PCT Filed: Jul. 23, 2012
(86) PCT No.: PCT/IB2012/053738

§ 371 Date: Jan. 16, 2014

(30) Foreign Application Priority Data
Jul. 25, 2011 (IN) 2098/MUM/2011

(51) Int. Cl. A61K 31/58; A61K 31/517; A61K 31/573; A61K 31/519; A61K 31/56

(52) U.S. Cl. CPC A61K 31/58; A61K 31/519; A61K 31/56; A61K 31/573; A61K 31/517
USPC 514/171

(57) ABSTRACT

The present patent application relates to a pharmaceutical composition comprising a transient receptor potential ankyrin-1 receptor (“TRPA1”) antagonist and a glucocorticoid.

### PRIORITY DOCUMENT

This patent application claims priority to Indian Provisional Patent Application number 2098/MUM/2011 (filed on Jul. 25, 2011), the contents of which are incorporated by reference herein.

### TECHNICAL FIELD

The present patent application relates to a pharmaceutical composition comprising a transient receptor potential ankyrin-1 receptor (“TRPA1”) antagonist and a steroid. Particularly, the application provides a pharmaceutical composition comprising a TRPA1 antagonist having IC₅₀ for inhibiting human TRPA1 receptor activity of less than 1 micromolar with respect to TRPA1 activity and a glucocorticoid; a process for preparing such composition; and its use in treating a respiratory disorder in a subject in need thereof.

### BACKGROUND

Respiratory disorders related to airway inflammation include a number of severe lung diseases including asthma and chronic obstructive pulmonary disease (“COPD”). The airways of asthmatic patients are infiltrated by inflammatory leukocytes, of which the eosinophil is believed to be the most prominent component. Inflammatory sensitization of airway neurons is believed to increase nasal and cough sensitivity, heighten the sense of irritation, and promote fluid secretion, airway narrowing, and bronchoconstriction.

TRPA1 receptor activation in the airways by exogenous noxious stimuli, including cold temperatures (generally, less than about 17° C.), pungent natural compounds (e.g., mustard, cinnamon and garlic), tobacco smoke, tear gas and environmental irritants as well as by endogenous biochemical mediators released during inflammation, is supposed to be one of the mechanisms for neurogenic inflammation in the airways. Neurogenic inflammation is an important component of chronic airway diseases like COPD and asthma.

PCT Application Publication Nos. viz., WO 2004/055054, WO 2005/089206, WO 2007/073505, WO 2008/0949099, WO 2009/089082, WO 2009/002933 WO 2009/158719, WO 2009/144548, WO 2010/004390, WO 2010/109287, WO 2010/109334, WO 2010/109329, WO 2010/109328, WO 2010/125469 and WO 2010/004390 describe various transient receptor potential (“TRP”) receptor modulators.

Steroids, particularly glucocorticoids (also known as corticosteroids) are believed to be helpful in alleviating respiratory disorders. The glucocorticoids for respiratory disorders such as asthma are preferably administered by inhalation to reduce the incidence of steroid-related side effects linked to systemic delivery. The glucocorticoids are believed to block many of the inflammatory pathways activated in respiratory disorders. Glucocorticoids are currently believed to be the most effective available therapy for respiratory diseases (such as asthma).

The glucocorticoids for treatment or control of respiratory disorders include beclomethasone, dexamethasone, fluticasone, mometasone, triamcinolone, prednisone, prednisolone, methylprednisolone, budesonide, ciclesonide, and flunisolide or salts thereof.

Fluticasone propionate is chemically known as S-(fluoromethyl) 6α, 9-difluoro-11β, 17-dihydroxy-16α-methyl-3-oxoandrosta-1,4-diene-17β-carbothioate, 17-propionate. Fluticasone propionate is available commercially as FLOVENT® HFA (marketed by Glaxo) in the United States as 50 μg, 100 μg and 250 μg powder for inhalation. Fluticasone propionate is indicated for the maintenance treatment of asthma as prophylactic therapy. It is also indicated for patients requiring oral corticosteroid therapy for asthma.

Prednisolone acetate is chemically 11β 17, 21-trihydroxypregna-1,4-diene-3,20-dione 21-acetate. It is commercially available in the United States as FLO-PRED as 15 mg/5 mL oral suspension (marketed by Taro) and as oral syrup (5 mg/mL and 15 mg/mL). It is indicated in the treatment of severe or incapacitating allergic; dermatological diseases; pulmonary diseases; rheumatologic conditions as adjunctive therapy for short-term administration, among others.

Budesonide is chemically, (RS)-11b, 16a, 17, 21-Tetrahydroxypregna-1,4-diene-3, 20-dione cyclic 16, 17-acetal with butyraldehyde. Budesonide is provided as a mixture of two epimers (22R and 22S). It is available commercially as PLUMICORT FLEXHALER (marketed by AstraZeneca AB) in the United States in the strengths of 0.08 mg/inh and 0.16 mg/inh. It is indicated for the maintenance treatment of asthma as prophylactic therapy. It is also available commercially as 3 mg oral capsule as ENTOCORT EC (marketed by AstraZeneca AB). It is approved for the treatment of mild to moderate active Crohn's disease involving the ileum and/or the ascending colon. It is also approved for the maintenance of clinical remission of mild to moderate Crohn's disease involving the ileum and/or the ascending colon for up to 3 months.

There still exists a need for an effective therapeutic treatment for respiratory disorders like asthma and COPD.

### SUMMARY

The inventors of the present invention have invented a pharmaceutical composition comprising a TRPA1 antagonist and a glucocorticoid.

The inventors have surprisingly found that a TRPA1 antagonist and a glucocorticoid act synergistically in the treatment of respiratory disorders and are more effective and provide better therapeutic value than treatment with either active ingredient alone.

Thus, in an embodiment, the present invention relates to a pharmaceutical composition comprising:

a) a TRPA1 antagonist, and

b) a glucocorticoid.

In another embodiment, the present invention relates to a pharmaceutical composition comprising:
a) a TRPA1 antagonist having a human IC₅₀ value of less than 1 micromolar; and
b) a glucocorticoid.

Preferably, the TRPA1 antagonist of the present invention has a human IC₅₀ value of less than 500 nanomolar, or more preferably less than 250 nanomolar, as measured by a method described herein.

The glucocorticoid, as contemplated herein, including prednisolone, beclomethasone, dexamethasone, fluticasone, mometasone, triamcinolone, prednisone, methylprednisolone, budesonide, ciclesonide, and flunisolide or salts thereof may be present in the form of its isomers, polymorphs, and solvates, including hydrates, all of which are included in the scope of the invention. Preferably, the glucocorticoid includes fluticasone, prednisolone, budesonide or salts thereof.

In an embodiment, the present invention relates to a pharmaceutical composition comprising synergistically effective amount of a TRPA1 antagonist having an IC₅₀ for inhibiting human TRPA1 receptor activity of less than 1 micromolar, and a glucocorticoid. Preferably, the TRPA1 antagonist of the present invention has an IC₅₀ for inhibiting human TRPA1 receptor activity of less than 500 nanomolar, or more preferably less than 250 nanomolar, as measured by a method described herein.

In another embodiment, the present invention relates to a pharmaceutical composition comprising synergistically effective amount of a TRPA1 antagonist having an IC₅₀ for inhibiting human TRPA1 receptor activity of less than 1 micromolar having structure of formulae: (XII) or (D)

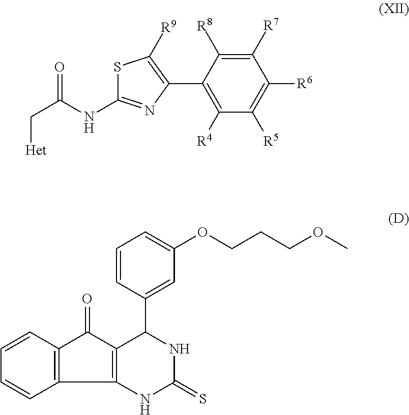

or a pharmaceutically-acceptable salt thereof, wherein, ‘Het’ is selected from the group consisting of

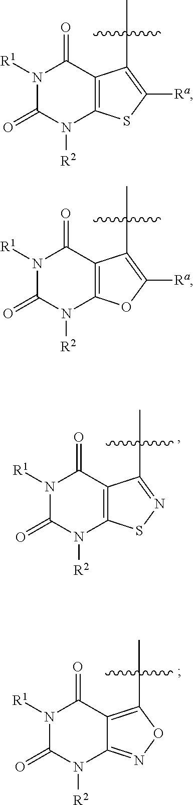

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl and a glucocorticoid.

In yet another embodiment, the present invention relates to a pharmaceutical composition comprising synergistically effective amount of a TRPA1 antagonist having structure of formula:

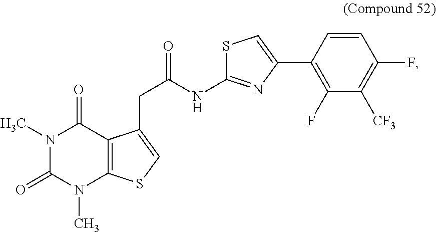 [SMILES: Cn1c(=O)c2c(CC(=O)Nc3nc(-c4ccc(F)c(C(F)(F)F)c4F)cs3)csc2n(C)c1=O]

and a glucocorticoid.

In another embodiment, there is provided a pharmaceutical composition comprising synergistically effective amount of a TRPA1 antagonist having an IC₅₀ for inhibiting human TRPA1 receptor activity of less than 1 micromolar and a glucocorticoid in a weight ratio ranging from about 1:0.001 to about 1:5000.

In an embodiment, the present invention relates to a method of treating a respiratory disorder in a subject in need thereof, said method comprising administering to the subject the pharmaceutical composition comprising synergistically effective amount of a TRPA1 antagonist having an IC₅₀ for inhibiting human TRPA1 receptor activity of less than 1 micromolar and a glucocorticoid. In an aspect of this embodiment, the TRPA1 antagonist has an IC₅₀ for inhibiting human TRPA1 receptor activity of less than 1 micromolar having structure of formulae: (XII) or (D)

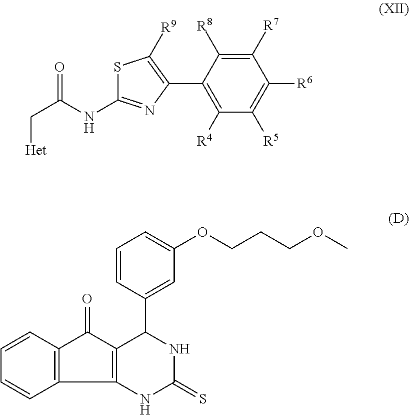

or a pharmaceutically-acceptable salt thereof, wherein, ‘Het’ is selected from the group consisting of

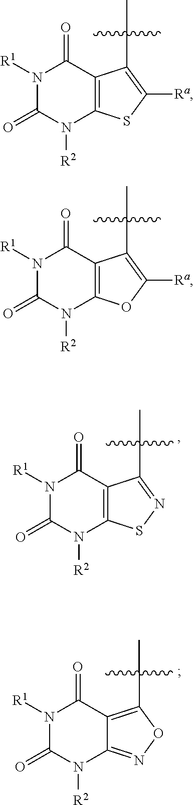

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

The respiratory disorder, in the context of present invention, includes but is not limited to asthma, emphysema, bronchitis, COPD, sinusitis, respiratory depression, reactive airways dysfunction syndrome (RADS), acute respiratory distress syndrome (ARDS), irritant induced asthma, occupational asthma, sensory hyper-reactivity, airway (or pulmonary) inflammation, multiple chemical sensitivity, and aid in smoking cessation therapy.

In a further embodiment, the present invention relates to a method of treating a respiratory disorder in a subject in need thereof, said method comprising administering the subject a pharmaceutical composition comprising synergistically effective amount of a TRPA1 antagonist having an IC₅₀ for inhibiting human TRPA1 receptor activity of less than 1 micromolar and glucocorticoid selected from a group consisting of prednisolone, beclomethasone, dexamethasone, fluticasone, mometasone, triamcinolone, prednisone, methylprednisolone, budesonide, ciclesonide, flunisolide or salts thereof. In an aspect of the embodiment, the glucocorticoid is fluticasone, prednisolone, budesonide or salts thereof.

In a further embodiment, the present invention relates to use of synergistically effective amount of a TRPA1 antagonist having an IC₅₀ for inhibiting human TRPA1 receptor activity of less than 1 micromolar and a glucocorticoid in the preparation of a pharmaceutical composition of the present invention for the treatment of a respiratory disorder in a subject in need thereof. In an aspect of this embodiment, the TRPA1 antagonist has an IC₅₀ for inhibiting human TRPA1 receptor activity of less than 1 micromolar having structure of formulae: (XII) or (D)

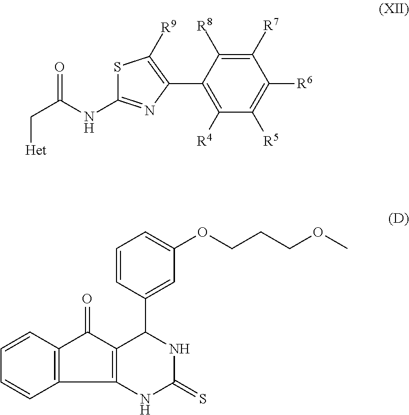

or a pharmaceutically-acceptable salt thereof, wherein, ‘Het’ is selected from the group consisting of

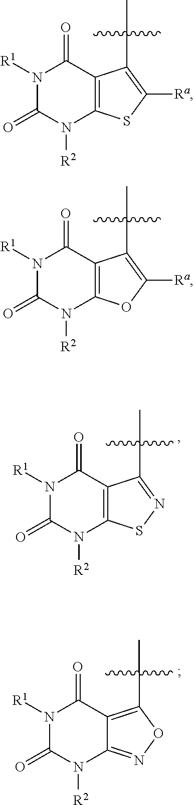

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In a further embodiment, the present invention relates to a pharmaceutical composition comprising synergistically effective amount of a TRPA1 antagonist having an IC₅₀ for inhibiting human TRPA1 receptor activity of less than 1 micromolar and a glucocorticoid for the treatment of a respiratory disorder in a subject in need thereof.

In an embodiment, the present invention relates to a pharmaceutical composition comprising synergistically effective amount of a TRPA1 antagonist having structure of formula:

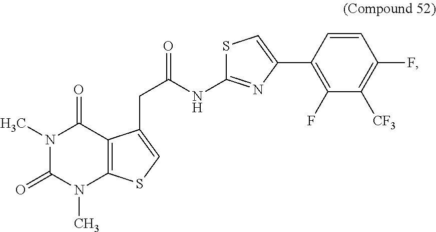 [SMILES: Cn1c(=O)c2c(CC(=O)Nc3nc(-c4ccc(F)c(C(F)(F)F)c4F)cs3)csc2n(C)c1=O]

and a glucocorticoid selected from a group consisting of prednisolone, beclomethasone, dexamethasone, fluticasone, mometasone, triamcinolone, prednisone, methylprednisolone, budesonide, ciclesonide, flunisolide or salts thereof. In an aspect of this embodiment, the pharmaceutical composition is a fixed dose combination.

In another aspect of this embodiment, the composition is for oral administration and the TRPA1 antagonist and the glucocorticoid are present in a weight ratio ranging from about 1:0.001 to about 1:100. In an aspect of the embodiment, the TRPA1 antagonist and the glucocorticoid are present in a weight ratio ranging from about 1:0.003 to about 1:15. The glucocorticoid for oral administration includes prednisolone, budesonide or salts thereof.

In yet another aspect of this embodiment, the composition is for inhalation administration and the TRPA1 antagonist and the glucocorticoid are present in a weight ratio ranging from about 1:0.001 to about 1:5000. In an aspect of the embodiment, the TRPA1 antagonist and the glucocorticoid are present in a weight ratio ranging from about 1:0.0025 to about 1:3200. The glucocorticoid for inhalation administration includes fluticaone, prednisolone, budesonide or salts thereof.

In an embodiment, the present invention relates to a method of treating a respiratory disorder in a subject in need thereof, said method comprising administering to the subject the pharmaceutical composition comprising synergistically effective amount of a TRPA1 antagonist having structure of formula:

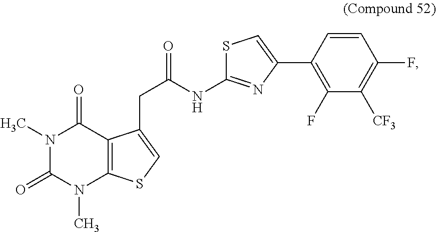 [SMILES: Cn1c(=O)c2c(CC(=O)Nc3nc(-c4ccc(F)c(C(F)(F)F)c4F)cs3)csc2n(C)c1=O]

and a glucocorticoid. In an aspect of this embodiment, the glucocorticoid is selected from a group consisting of prednisolone, beclomethasone, dexamethasone, fluticasone, mometasone, triamcinolone, prednisone, methylprednisolone, budesonide, ciclesonide, flunisolide or salts thereof.

In an embodiment, the present invention relates to a method of treating a respiratory disorder by reducing eosinophils count and/or increasing FEV1 value in a subject in need thereof, said method comprising administering to the subject the pharmaceutical composition comprising synergistically effective amount of a TRPA1 antagonist having structure of formula:

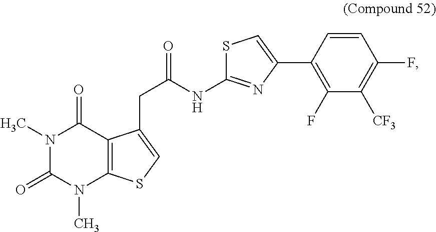 [SMILES: Cn1c(=O)c2c(CC(=O)Nc3nc(-c4ccc(F)c(C(F)(F)F)c4F)cs3)csc2n(C)c1=O]

and a glucocorticoid, thereby reducing said eosinophil count and/or increasing FEV1 value in said subject.

In an embodiment, the present invention relates to a method of treating a respiratory disorder by reducing airway inflammation in a subject in need thereof, said method comprising administering to the subject the pharmaceutical composition comprising synergistically effective amount of a TRPA1 antagonist having structure of formula:

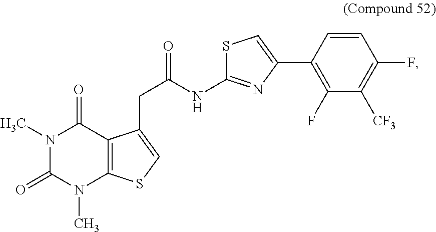 [SMILES: Cn1c(=O)c2c(CC(=O)Nc3nc(-c4ccc(F)c(C(F)(F)F)c4F)cs3)csc2n(C)c1=O]

and a glucocorticoid, thereby reducing said airway inflammation.

In an aspect of this embodiment, the glucocorticoid is selected from a group consisting of prednisolone, beclomethasone, dexamethasone, fluticasone, mometasone, triamcinolone, prednisone, methylprednisolone, budesonide, ciclesonide, flunisolide or salts thereof. In another aspect of this embodiment, the respiratory disorder is asthma.

In an embodiment, the present invention relates to a method of reducing eosinophils count and/or increasing FEV1 value in a subject in need thereof, said method comprising administering to the subject the pharmaceutical composition comprising synergistically effective amount of a TRPA1 antagonist having structure of formula:

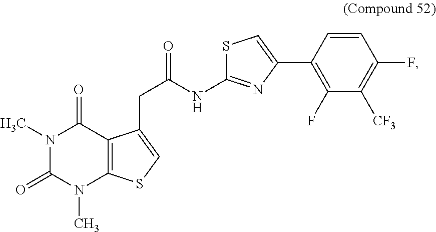 [SMILES: Cn1c(=O)c2c(CC(=O)Nc3nc(-c4ccc(F)c(C(F)(F)F)c4F)cs3)csc2n(C)c1=O]

and a glucocorticoid, thereby reducing said eosinophil count and/or increasing FEV1 value in said subject.

In an embodiment, the present invention relates to a method of reducing airway inflammation in a subject in need thereof, said method comprising administering to the subject the pharmaceutical composition comprising synergistically effective amount of a TRPA1 antagonist having structure of formula:

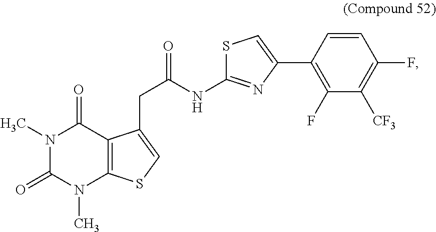 [SMILES: Cn1c(=O)c2c(CC(=O)Nc3nc(-c4ccc(F)c(C(F)(F)F)c4F)cs3)csc2n(C)c1=O]

and a glucocorticoid, thereby reducing said airway inflammation in said subject.

In an aspect of this embodiment, the glucocorticoid is selected from a group consisting of prednisolone, beclomethasone, dexamethasone, fluticasone, mometasone, triamcinolone, prednisone, methylprednisolone, budesonide, ciclesonide, flunisolide or salts thereof.

In another embodiment, the present invention relates to use of synergistically effective amount of a TRPA1 antagonist having structure of formula:

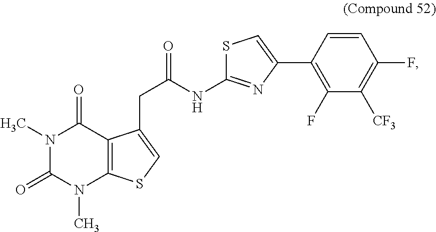 [SMILES: Cn1c(=O)c2c(CC(=O)Nc3nc(-c4ccc(F)c(C(F)(F)F)c4F)cs3)csc2n(C)c1=O]

and glucocorticoids in the preparation of a pharmaceutical composition of the present invention for the treatment of a respiratory disorder in a subject in need thereof. In an aspect of this embodiment, the glucocorticoid is selected from a group consisting of prednisolone, beclomethasone, dexamethasone, fluticasone, mometasone, triamcinolone, prednisone, methylprednisolone, budesonide, ciclesonide, flunisolide or salts thereof.

In a further embodiment, the present invention relates to a pharmaceutical composition comprising synergistically effective amount of a TRPA1 antagonist having structure of formula:

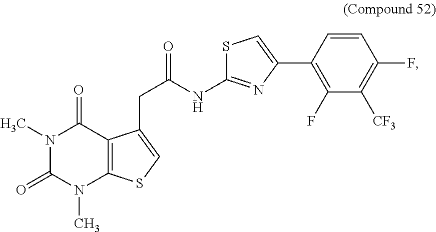 [SMILES: Cn1c(=O)c2c(CC(=O)Nc3nc(-c4ccc(F)c(C(F)(F)F)c4F)cs3)csc2n(C)c1=O]

and a glucocorticoid for the treatment of a respiratory disorder in a subject in need thereof.

### BRIEF DESCRIPTION OF THE DRAWINGS

FIG. 1 is a bar graph showing the effect of Compound 52 and prednisolone on total cells in BALf in mouse model of asthma.

FIG. 2 is a bar graph showing the effect of Compound 52 and prednisolone on total eosinophil count in BALf in mouse model of asthma.

FIG. 3 is a bar graph showing the effect of Compound 52 and fluticasone on total cells in BALf in Brown Norway rat model of asthma.

FIG. 4 is a bar graph showing the effect of Compound 52 and fluticasone on total eosinophil count in BALf in Brown Norway rat model of asthma.

FIG. 5 is a bar graph showing the effect of Compound 52 and budesonide on total cells in BALf in mouse model of asthma.

FIG. 6 is a bar graph showing the effect of Compound 52 and budesonide on total eosinophil count in BALf in mouse model of asthma.

### DETAILED DESCRIPTION

### Definitions

The terms used herein are defined as follows. If a definition set forth in the present application and a definition set forth earlier in a provisional application from which priority is claimed are in conflict, the definition in the present application shall control the meaning of the terms.

The term “effective amount” or “therapeutically effective amount” denotes an amount of an active ingredient that, when administered to a subject for treating a respiratory disorder, produces an intended therapeutic benefit in a subject in need thereof. The effective amount of TRPA1 antagonist as described herein ranges from about 0.1 μg/kg to about 20 mg/kg, and preferably from about 1 μg/kg to about 15 mg/kg. The therapeutically effective amount of fluticasone or its salt to be administered per day ranges from about 10 μg to about 5 mg, and preferably from about 50 μg to about 3 mg, and more preferably from about 100 μg to about 2 mg. The therapeutically effective amount of prednisolone or its salt to be administered per day ranges from about 1 mg to about 100 mg; and preferably from about 2 mg to about 75 mg; and more preferably from about 5 mg to about 60 mg. The therapeutically effective amount of budesonide or its salt to be administered per day ranges from about 0.01 mg to about 20 mg; and preferably from about 0.05 mg to about 10 mg; and more preferably from about 0.09 mg to about 9 mg. The therapeutically effective ranges of actives are given as above, although larger or smaller amount are not excluded if they fall within the scope of the definition of this paragraph.

The term “active ingredient” (used interchangeably with “active” or “active substance” or “drug”) as used herein includes a TRPA1 antagonist, a glucocorticoid or a pharmaceutically acceptable salt thereof. Preferably, the active ingredient includes TRPA1 antagonist having a human IC₅₀ value of less than 1 micromolar, fluticasone or prednisolone or budesonide or its salt.

The IC₅₀ value is believed to be measure of the effectiveness of a compound in inhibiting biological or biochemical function. This quantitative measure generally indicates molar concentration of a particular compound (or substance) is needed to inhibit a given biological process by half. In other words, it is the half maximal (50%) inhibitory concentration (IC) of the compound. The IC₅₀ of a drug compound (or active substance) can be determined by constructing a concentration-response curve so as to examine the effect of different concentrations of antagonist on reversing agonist activity. IC₅₀ values can be calculated for a given antagonist by determining the concentration needed to inhibit half of the maximum biological response of the agonist. IC₅₀ values can be used to compare the potency of two antagonists.

By “salt” or “pharmaceutically acceptable salt”, it is meant those salts and esters which are, within the scope of sound medical judgment, suitable for use in contact with the tissues of humans and lower animals without undue toxicity, irritation, and allergic response, commensurate with a reasonable benefit to risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, propionate, acetate and lauryl sulphate salts. Representative alkali or alkaline earth metal salts include the sodium, calcium, potassium and magnesium salts.

The term “treating” or “treatment” as used herein also covers the prophylaxis, mitigation, prevention, amelioration, or suppression of a disorder modulated by the TRPA1 receptor, or the glucocorticoid receptor, or by a combination of the two in a mammal.

The respiratory disorder, in the context of present invention, includes but is not limited to asthma, emphysema, bronchitis, COPD, sinusitis, respiratory depression, reactive airways dysfunction syndrome (RADS), acute respiratory distress syndrome (ARDS), irritant induced asthma, occupational asthma, sensory hyper-reactivity, airway (or pulmonary) inflammation, multiple chemical sensitivity, and aid in smoking cessation therapy.

The term “subject” includes mammals like human and other animals, such as domestic animals (e.g., household pets including cats and dogs) and non-domestic animals (such as wildlife). Preferably, the subject is a human.

By “pharmaceutically acceptable excipients”, it is meant any of the components of a pharmaceutical composition other than the actives and which are approved by regulatory authorities or are generally regarded as safe for human or animal use.

### Combinations

The inventors of the present invention have invented a pharmaceutical composition comprising a TRPA1 antagonist and a glucocorticoid.

The inventors have surprisingly found that a TRPA1 antagonist and a glucocorticoid act synergistically in the treatment of respiratory disorders, and are more effective and provide better therapeutic value than treatment with either active ingredient alone.

Thus, in an embodiment, the present invention relates to a pharmaceutical composition comprising:

a) a TRPA1 antagonist, and

b) a glucocorticoid.

In another embodiment, the present invention relates to a pharmaceutical composition comprising:
a) a TRPA1 antagonist having a human IC₅₀ value of less than 1 micromolar; and
b) a glucocorticoid.

Preferably, the TRPA1 antagonist of the present invention has a human IC₅₀ value of less than 500 nanomolar, or more preferably less than 250 nanomolar, as measured by a method described herein.

In an aspect, TRPA1 antagonists useful in the context of the invention, are selected from one of the following formulae: (A) or (B) or (C) or (D)

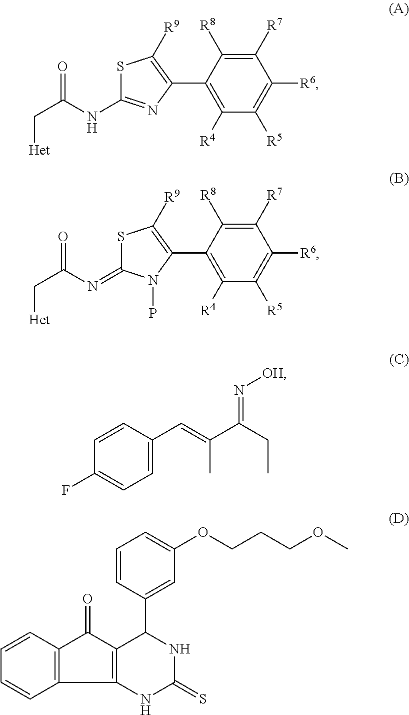

or a pharmaceutically-acceptable salt thereof, wherein, ‘Het’ is selected from the group consisting of

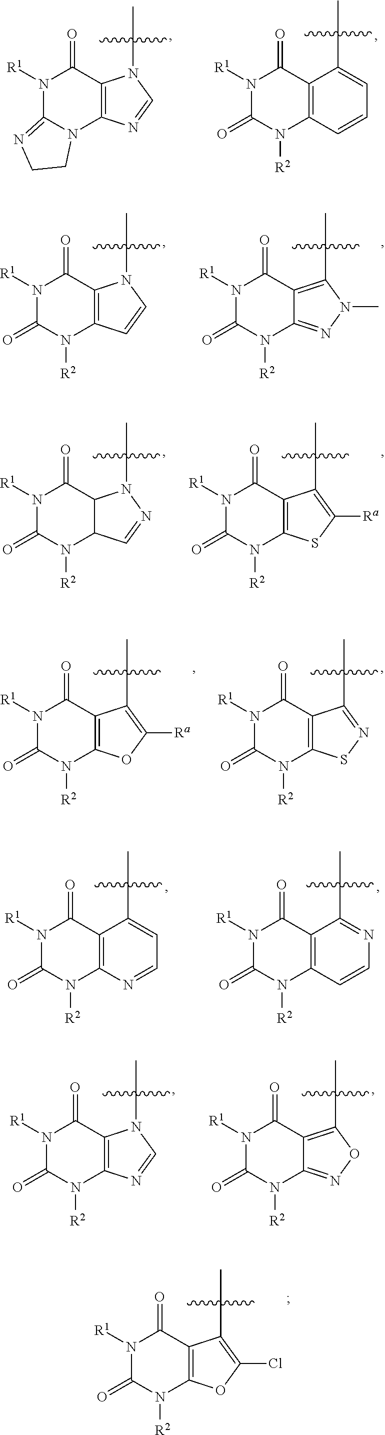

P is selected from

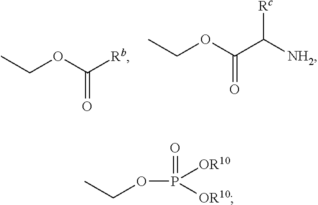

R¹, R² and R^{a}, which may be the same or different, are each independently hydrogen or (C₁-C₄) alkyl;

R^{b} and R^{c} independently selected from hydrogen, substituted or unsubstituted alkyl arylalkyl, amino acid and heterocyclic ring;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

R¹⁰ is selected from hydrogen, alkyl, arylalkyl and pharmaceutically acceptable cation.

In one aspect, TRPA1 antagonists useful in the context of the invention are selected from those compounds generically or specifically disclosed in WO2009144548. Accordingly, a TRPA1 antagonist useful in the context of the invention has the formula (I):

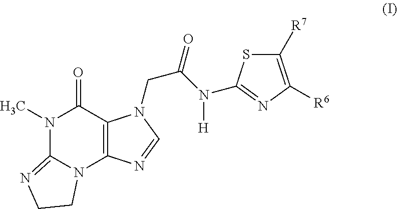

or a pharmaceutically acceptable salt thereof,
wherein,

R⁶ represents hydrogen, substituted or unsubstituted alkyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl;

R⁷ independently represents hydrogen or alkyl.

Few representative TRPA1 antagonists useful in the methods of the invention are mentioned below:

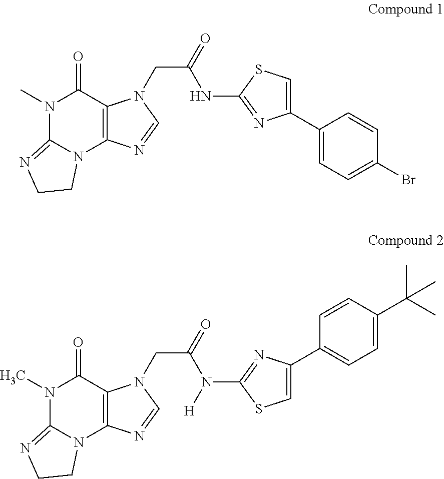 [SMILES: CN1C(=O)c2c(ncn2CC(=O)Nc2nc(-c3ccc(Br)cc3)cs2)N2CCN=C12 ; CN1C(=O)c2c(ncn2CC(=O)Nc2nc(-c3ccc(C(C)(C)C)cc3)cs2)N2CCN=C12]

The preparation of above said compounds is described in WO2009144548.

In another aspect, TRPA1 antagonists useful in the context of the invention are selected from those compounds generically or specifically disclosed in WO2010004390. Accordingly, TRPA1 antagonist useful in the context of the invention has the formula (II):

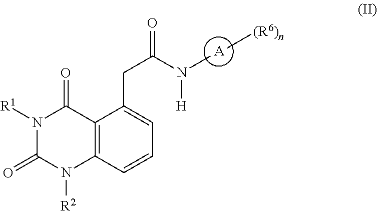

or pharmaceutically acceptable salts thereof,
wherein,

at each occurrence R¹ and R² is independently selected from hydrogen, hydroxyl, substituted or unsubstituted alkyl, substituted or unsubstituted alkenyl, substituted or unsubstituted alkynyl, substituted or unsubstituted cycloalkyl, substituted or unsubstituted cycloalkylalkyl, (CR^{x}R^{y})ₙOR^{x}, COR^{x}, COOR^{x}, CONR^{x}R^{y}, SO₂NR^{x}R^{y}, NR^{x}R^{y}, NR^{x}(CR^{x}R^{y})ₙOR^{x}, NR^{x}(CR^{x}R^{y})ₙCN(CH₂)ₙNR^{x}R^{y}, (CH₂)ₙCHR^{x}R^{y}, (CR^{x}R^{y})NR^{x}R^{y}, NR^{x}(CR^{x}R^{y})ₙCONR^{x}R^{y}, (CH₂)ₙNHCOR^{x} and (CH₂)ₙNH(CH₂)ₙSO₂R^{x}, (CH₂)ₙNHSO₂R^{x};

R^{x} and R^{y} are independently selected from hydrogen, hydroxyl, halogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl;

R^{x} and R^{y} may be joined together to form an optionally substituted 3 to 7 membered saturated, unsaturated or partially saturated cyclic ring, which may optionally include at least two heteroatoms selected from O, NR^{a} or S;

ring A is selected from phenyl, pyridinyl, pyrazolyl, thiazolyl and thiadiazolyl;

each occurrence of R⁶ is independently hydrogen, cyano, nitro, —NR^{x}R^{y}, halogen, hydroxyl, haloalkyl, haloalkoxy, cycloalkylalkoxy, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl,

R^{x} and R^{y} are independently selected from hydrogen, hydroxyl, halogen, substituted or unsubstituted alkyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, and substituted or unsubstituted heteroarylalkyl;

at each occurrence of ‘n’ is independently selected from 1 to 5.

According to one aspect, specifically provided are compounds of the formula (IIa)

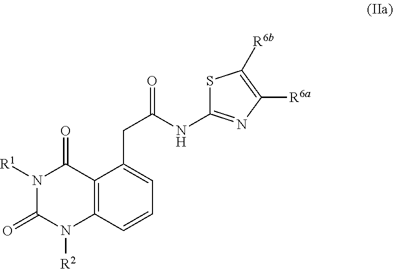

or pharmaceutically acceptable salts thereof,
wherein,

R¹ and R² are as defined above for the compound of formula (II);

R^{6a} and R^{6b} are independently selected from hydrogen, cyano, nitro, —NR^{x}R^{y}, halogen, hydroxyl, haloalkyl, haloalkoxy, cycloalkylalkoxy, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic ring and substituted or unsubstituted heterocyclylalkyl, —C(O)OR^{x}, —OR^{x}, —C(O)NR^{x}R^{y}, —C(O)R^{x}, —SO₂R^{x}, —SO₂—NR^{x}R^{y}.

Few representative TRPA1 antagonists useful in the context of the invention are mentioned below:

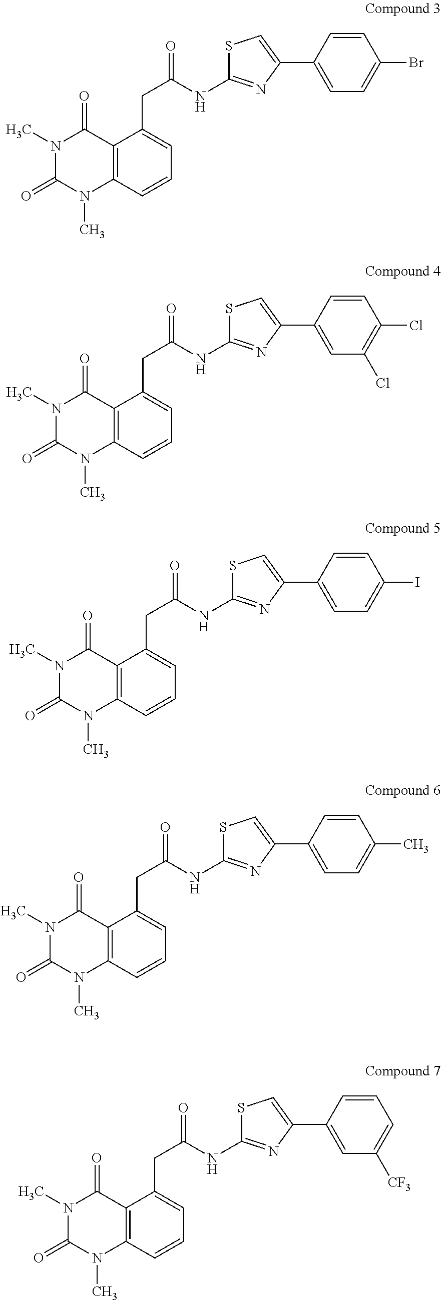 [SMILES: Cn1c(=O)c2c(CC(=O)Nc3nc(-c4ccc(Br)cc4)cs3)cccc2n(C)c1=O ; Cn1c(=O)c2c(CC(=O)Nc3nc(-c4ccc(Cl)c(Cl)c4)cs3)cccc2n(C)c1=O ; Cn1c(=O)c2c(CC(=O)Nc3nc(-c4ccc(I)cc4)cs3)cccc2n(C)c1=O ; Cc1ccc(-c2csc(NC(=O)Cc3cccc4c3c(=O)n(C)c(=O)n4C)n2)cc1 ; Cn1c(=O)c2c(CC(=O)Nc3nc(-c4cccc(C(F)(F)F)c4)cs3)cccc2n(C)c1=O]

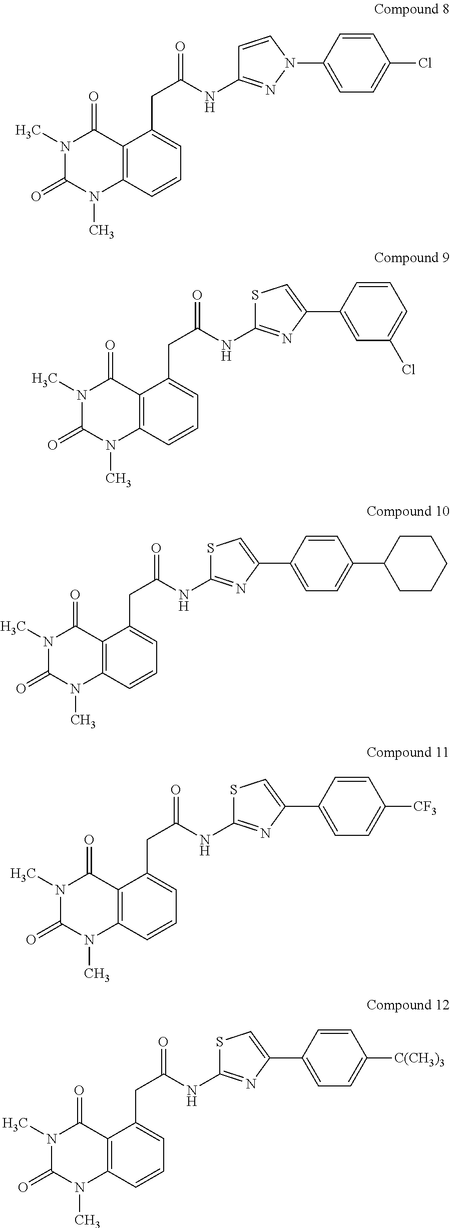 [SMILES: Cn1c(=O)c2c(CC(=O)Nc3ccn(-c4ccc(Cl)cc4)n3)cccc2n(C)c1=O ; Cn1c(=O)c2c(CC(=O)Nc3nc(-c4cccc(Cl)c4)cs3)cccc2n(C)c1=O ; Cn1c(=O)c2c(CC(=O)Nc3nc(-c4ccc(C5CCCCC5)cc4)cs3)cccc2n(C)c1=O ; Cn1c(=O)c2c(CC(=O)Nc3nc(-c4ccc(C(F)(F)F)cc4)cs3)cccc2n(C)c1=O ; Cn1c(=O)c2c(CC(=O)Nc3nc(-c4ccc(C(C)(C)C)cc4)cs3)cccc2n(C)c1=O]

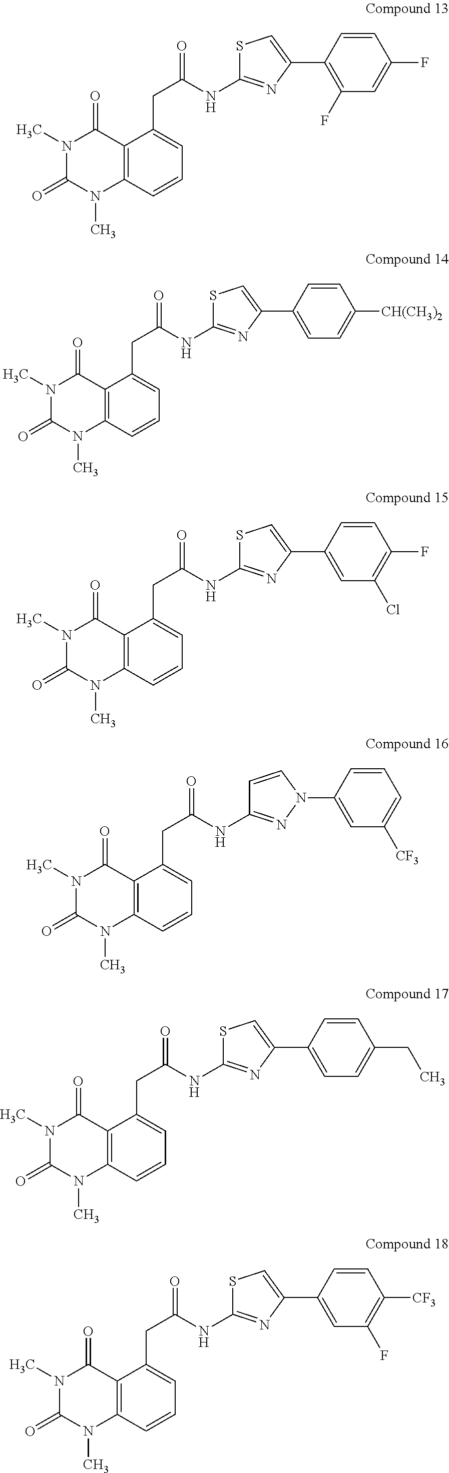 [SMILES: Cn1c(=O)c2c(CC(=O)Nc3nc(-c4ccc(F)cc4F)cs3)cccc2n(C)c1=O ; CC(C)c1ccc(-c2csc(NC(=O)Cc3cccc4c3c(=O)n(C)c(=O)n4C)n2)cc1 ; Cn1c(=O)c2c(CC(=O)Nc3nc(-c4ccc(F)c(Cl)c4)cs3)cccc2n(C)c1=O ; Cn1c(=O)c2c(CC(=O)Nc3ccn(-c4cccc(C(F)(F)F)c4)n3)cccc2n(C)c1=O ; CCc1ccc(-c2csc(NC(=O)Cc3cccc4c3c(=O)n(C)c(=O)n4C)n2)cc1 ; Cn1c(=O)c2c(CC(=O)Nc3nc(-c4ccc(C(F)(F)F)c(F)c4)cs3)cccc2n(C)c1=O]

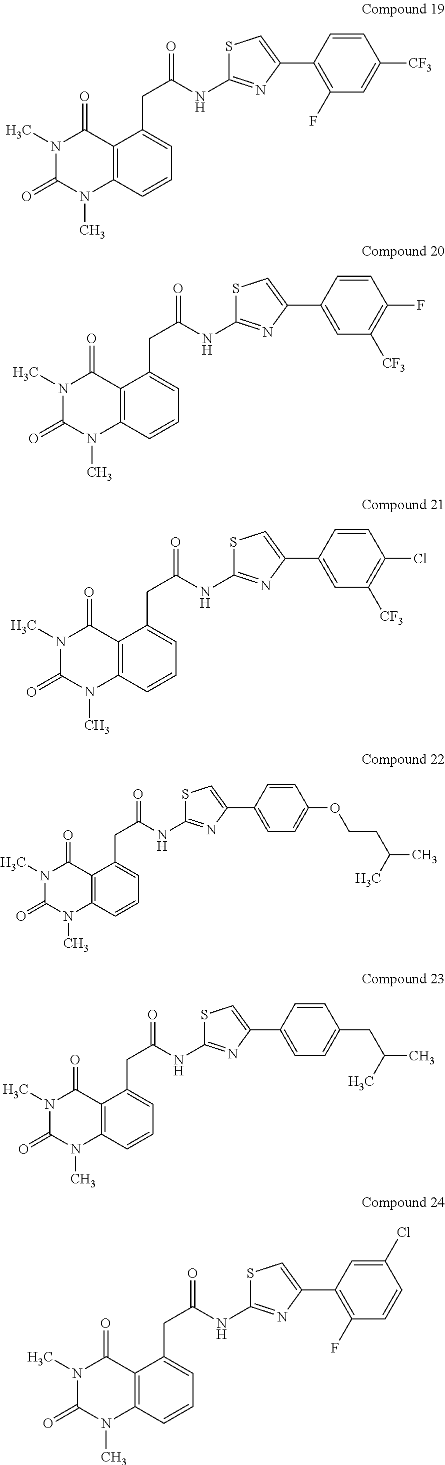 [SMILES: Cn1c(=O)c2c(CC(=O)Nc3nc(-c4ccc(C(F)(F)F)cc4F)cs3)cccc2n(C)c1=O ; Cn1c(=O)c2c(CC(=O)Nc3nc(-c4ccc(F)c(C(F)(F)F)c4)cs3)cccc2n(C)c1=O ; Cn1c(=O)c2c(CC(=O)Nc3nc(-c4ccc(Cl)c(C(F)(F)F)c4)cs3)cccc2n(C)c1=O ; CC(C)CCOc1ccc(-c2csc(NC(=O)Cc3cccc4c3c(=O)n(C)c(=O)n4C)n2)cc1 ; CC(C)Cc1ccc(-c2csc(NC(=O)Cc3cccc4c3c(=O)n(C)c(=O)n4C)n2)cc1 ; Cn1c(=O)c2c(CC(=O)Nc3nc(-c4cc(Cl)ccc4F)cs3)cccc2n(C)c1=O]

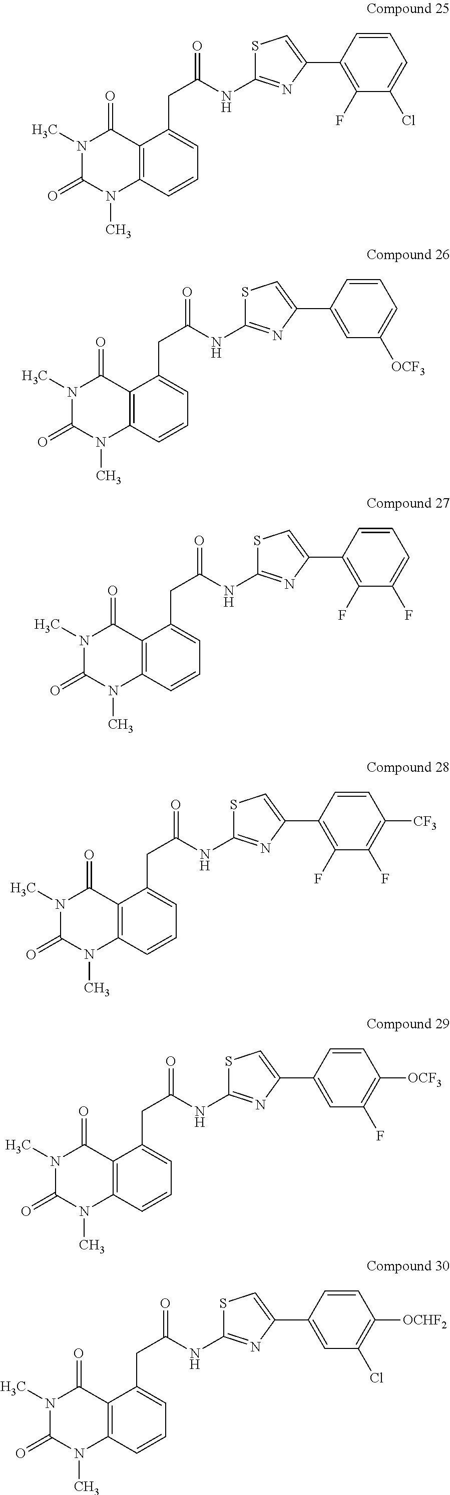 [SMILES: Cn1c(=O)c2c(CC(=O)Nc3nc(-c4cccc(Cl)c4F)cs3)cccc2n(C)c1=O ; Cn1c(=O)c2c(CC(=O)Nc3nc(-c4cccc(OC(F)(F)F)c4)cs3)cccc2n(C)c1=O ; Cn1c(=O)c2c(CC(=O)Nc3nc(-c4cccc(F)c4F)cs3)cccc2n(C)c1=O ; Cn1c(=O)c2c(CC(=O)Nc3nc(-c4ccc(C(F)(F)F)c(F)c4F)cs3)cccc2n(C)c1=O ; Cn1c(=O)c2c(CC(=O)Nc3nc(-c4ccc(OC(F)(F)F)c(F)c4)cs3)cccc2n(C)c1=O ; Cn1c(=O)c2c(CC(=O)Nc3nc(-c4ccc(OC(F)F)c(Cl)c4)cs3)cccc2n(C)c1=O]

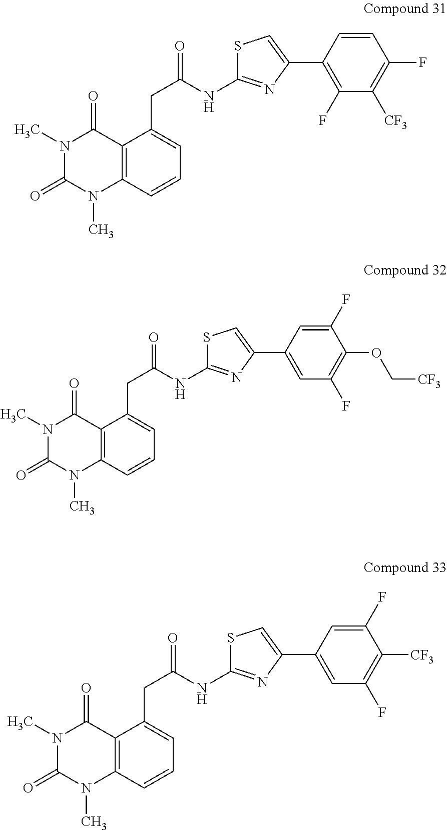 [SMILES: Cn1c(=O)c2c(CC(=O)Nc3nc(-c4ccc(F)c(C(F)(F)F)c4F)cs3)cccc2n(C)c1=O ; Cn1c(=O)c2c(CC(=O)Nc3nc(-c4cc(F)c(OCC(F)(F)F)c(F)c4)cs3)cccc2n(C)c1=O ; Cn1c(=O)c2c(CC(=O)Nc3nc(-c4cc(F)c(C(F)(F)F)c(F)c4)cs3)cccc2n(C)c1=O]

The preparation of above said compounds is described in WO2010004390.

In one aspect, TRPA1 antagonists useful in the context of the invention are selected from those compounds generically or specifically disclosed in WO2010109287. Accordingly, TRPA1 antagonist useful in the context of the invention has the formula (III):

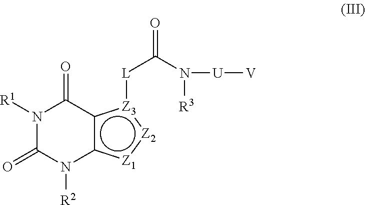

or a pharmaceutically acceptable salt thereof,
wherein,

Z₁ is NR^{a} or CR^{a};

Z₂ is NR^{b} or CR^{b};

Z₃ is N or C;

with the proviso that when Z₂ is CR^{b} then both Z₁ and Z₃ are not nitrogen at the same time;

at each occurrence, R^{a} and R^{b} which may be same or different, are independently selected from hydrogen, hydroxyl, cyano, halogen, substituted or unsubstituted alkyl, haloalkyl, alkenyl, alkynyl, cycloalkyl, cycloalkylalkyl, —(CR^{x}R^{y})ₙOR^{x}, —COR^{x}, —COOR^{x}, —CONR^{x}R^{y}, —S(O)ₙNR^{x}R^{y}, —NR^{x}R^{y}, —NR^{x}(CR^{x}R^{y})ₙOR^{x}, —(CH₂)ₙNR^{x}R^{Y}, —(CH₂)ₙCHR^{x}R^{y}, —(CH₂)NR^{x}R^{y}, —NR^{x}(CR^{x}R^{y})ₙCONR^{x}R^{y}, —(CH₂)ₙNHCOR^{x}, —(CH₂)ₙNH(CH₂)ₙSO₂R^{x} and (CH₂)ₙNHSO₂R^{x};

alternatively either of R^{a} or R^{b} is absent;

R¹ and R², which may be same or different, are independently selected from hydrogen, hydroxyl, substituted or unsubstituted alkyl, haloalkyl, alkenyl, alkynyl, cycloalkyl, cycloalkylalkyl, arylalkyl, (CR^{x}R^{y})ₙOR^{x}, COR^{x}, COOR^{x}, CONR^{x}R^{y}, (CH₂)ₙNR^{x}R^{y}, (CH₂)ₙCHR^{x}R^{y}, (CH₂)NR^{x}R^{y} and (CH₂)ₙNHCOR^{x};

R³ is selected from hydrogen, substituted or unsubstituted alkyl, alkenyl, haloalkyl, alkynyl, cycloalkyl, cycloalkylalkyl, cycloalkenyl;

L is a linker selected from —(CR^{x}R^{y})ₙ—, —O—(CR^{x}R^{y})ₙ—, —C(O)—, —NR^{x}—, —S(O)ₘNR^{x}—, —NR^{x}(CR^{x}R^{y})ₙ— and —S(O)ₘNR^{x}(CR^{x}R^{y})ₙ;

U is selected from substituted or unsubstituted aryl, substituted or unsubstituted five membered heterocycles selected from the group consisting of thiazole, isothiazole, oxazole, isoxazole, thiadiazole, oxadiazole, pyrazole, imidazole, furan, thiophene, pyroles, 1,2,3-triazoles and 1,2,4-triazole; and substituted or unsubstituted six membered heterocycles selected from the group consisting of pyrimidine, pyridine and pyridazine;

V is selected from hydrogen, cyano, nitro, —NR^{x}R^{y}, halogen, hydroxyl, substituted or unsubstituted alkyl, alkenyl, alkynyl, cycloalkyl, cycloalkylalkyl, cycloalkenyl, haloalkyl, haloalkoxy, cycloalkylalkoxy, aryl, arylalkyl, biaryl, heteroaryl, heteroarylalkyl, heterocyclic ring and heterocyclylalkyl, —C(O)OR^{x}, —OR^{x}, —C(O)NR^{x}R^{y}, —C(O)R^{x} and —SO₂NR^{x}R^{y}; or U and V together may form an optionally substituted 3 to 7 membered saturated or unsaturated cyclic ring, that may optionally include one or more heteroatoms selected from O, S and N;

at each occurrence, R^{x} and R^{y} are independently selected from the group consisting of hydrogen, hydroxyl, halogen, substituted or unsubstituted alkyl, alkenyl, alkynyl, cycloalkyl, cycloalkylalkyl, cycloalkenyl, aryl, arylalkyl, heteroaryl, heteroarylalkyl, heterocyclic ring and heterocyclylalkyl; and
at each occurrence ‘m’ and ‘n’ are independently selected from 0 to 2, both inclusive.

Few representative TRPA1 antagonists useful in the context of the invention are mentioned below:

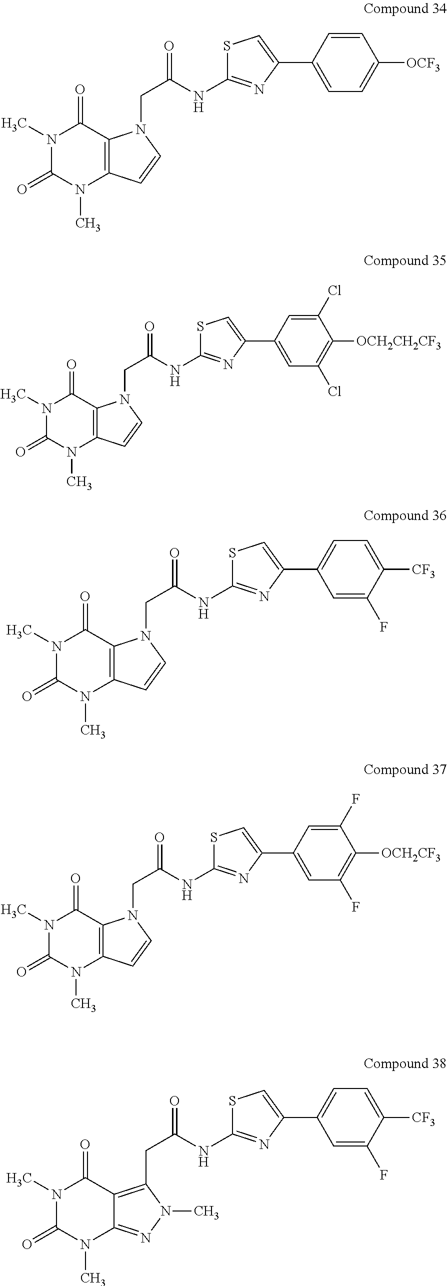

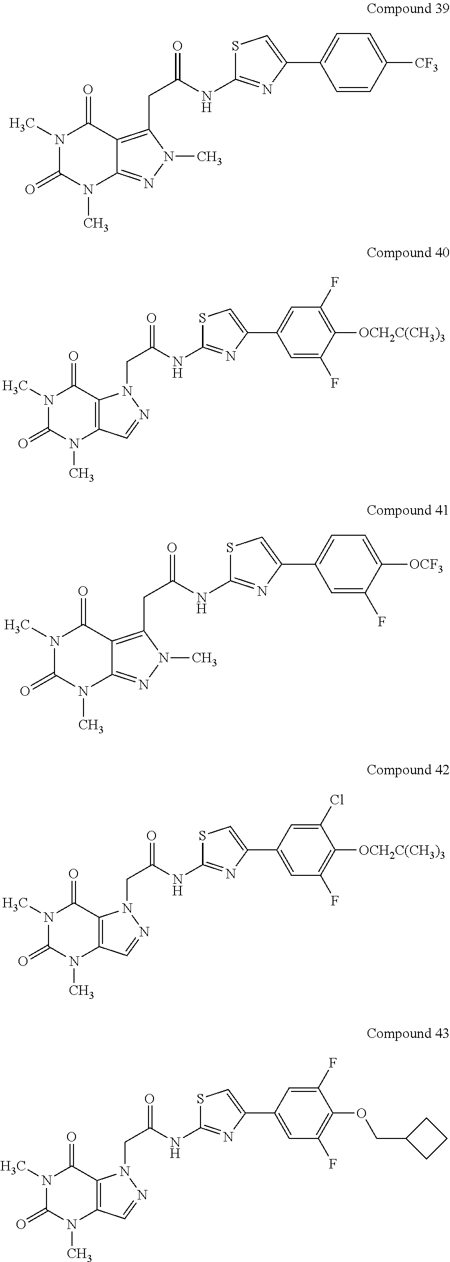

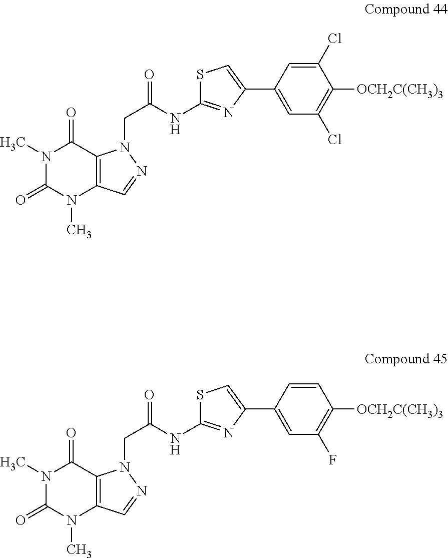

The preparation of above said compounds is described in WO2010109287.

In one aspect, TRPA1 antagonists useful in the context of the invention are selected from those compounds generically or specifically disclosed in WO 2010109334. Accordingly, TRPA1 antagonists useful in the context of the invention has the formula (IV)

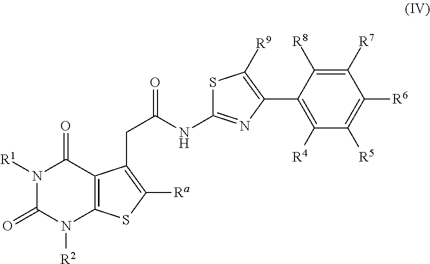

or a pharmaceutically-acceptable salt thereof.

wherein, R¹, R² and R^{a}, which may be the same or different, are each independently hydrogen or (C₁-C₄)alkyl;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

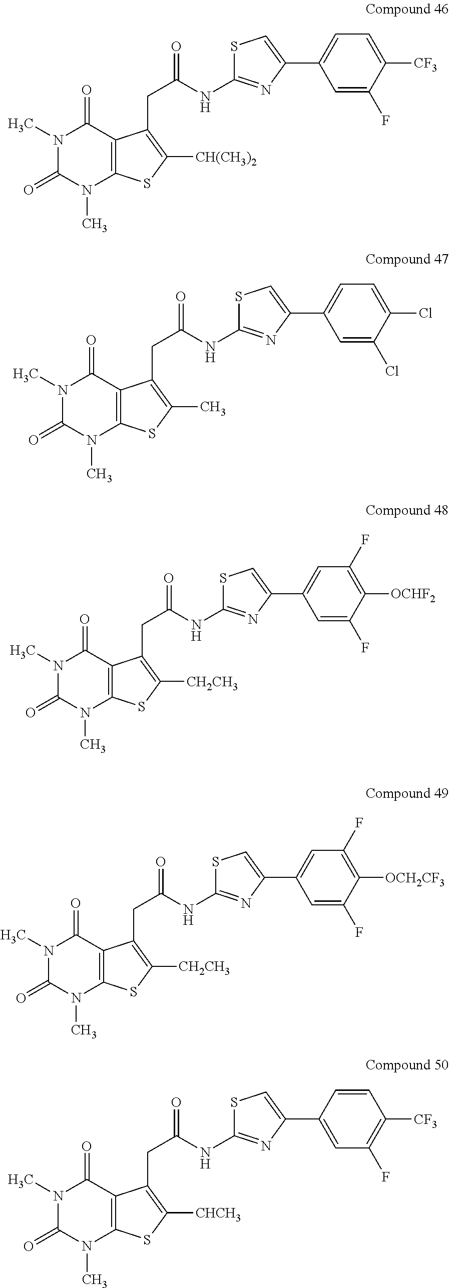

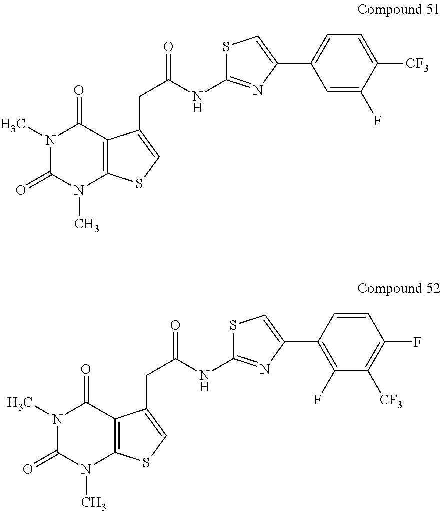 [SMILES: Cn1c(=O)c2c(CC(=O)Nc3nc(-c4ccc(C(F)(F)F)c(F)c4)cs3)csc2n(C)c1=O ; Cn1c(=O)c2c(CC(=O)Nc3nc(-c4ccc(F)c(C(F)(F)F)c4F)cs3)csc2n(C)c1=O]

The preparation of above said compounds is described in WO2010109334.

In one aspect, TRPA1 antagonists useful in the context of the invention are selected from those compounds generically or specifically disclosed in WO2010109329. Accordingly, TRPA1 antagonists useful in the context of the invention has the formula (V)

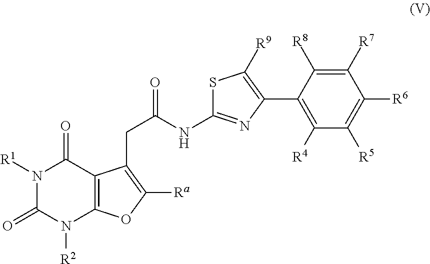

or a pharmaceutically acceptable salt thereof,
wherein, R¹, R² and R^{a} which may be the same or different, are each independently hydrogen or (C₁-C₄) alkyl; and

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

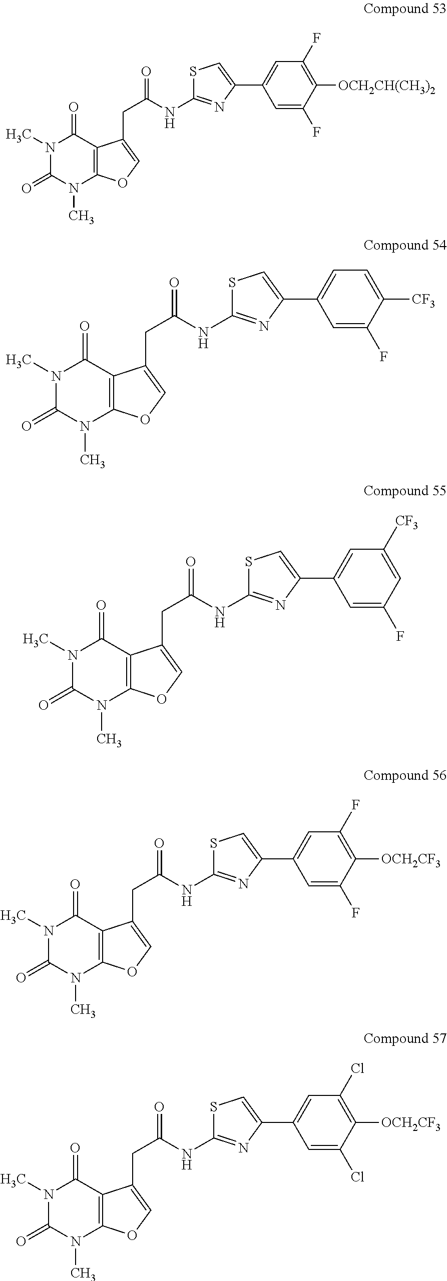

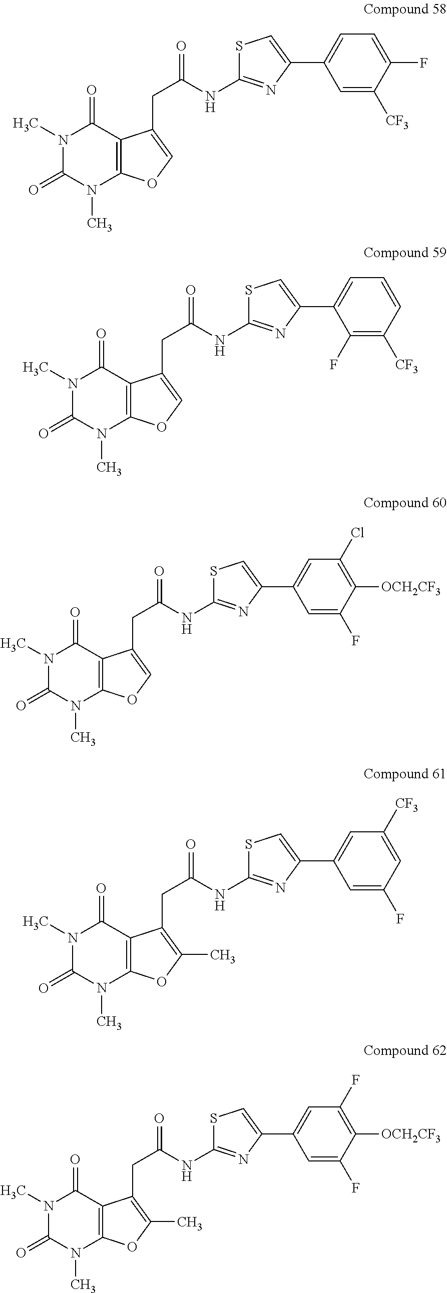

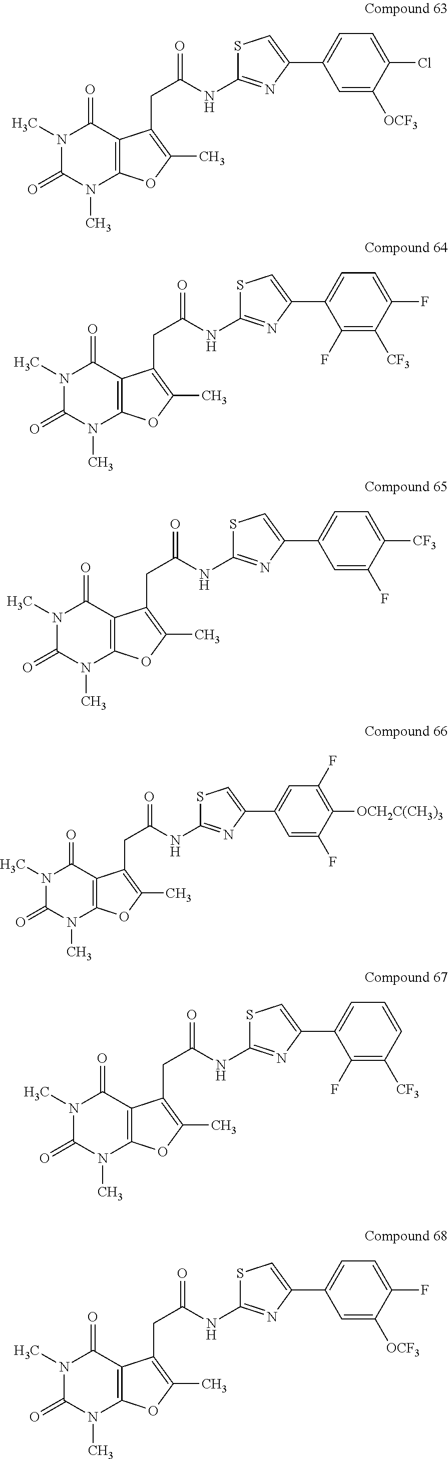

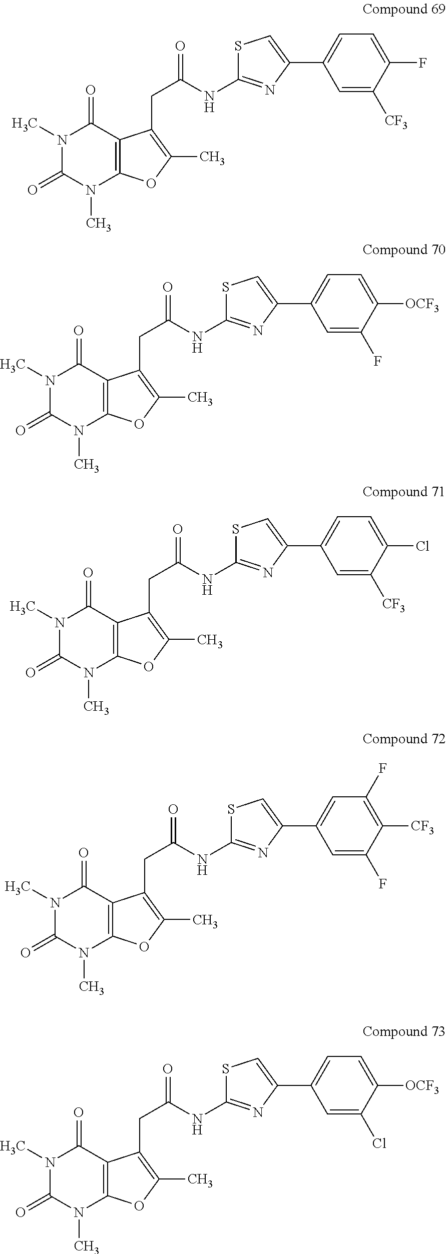 [SMILES: Cc1oc2c(c1CC(=O)Nc1nc(-c3ccc(F)c(C(F)(F)F)c3)cs1)c(=O)n(C)c(=O)n2C ; Cc1oc2c(c1CC(=O)Nc1nc(-c3ccc(OC(F)(F)F)c(F)c3)cs1)c(=O)n(C)c(=O)n2C ; Cc1oc2c(c1CC(=O)Nc1nc(-c3ccc(Cl)c(C(F)(F)F)c3)cs1)c(=O)n(C)c(=O)n2C ; Cc1oc2c(c1CC(=O)Nc1nc(-c3cc(F)c(C(F)(F)F)c(F)c3)cs1)c(=O)n(C)c(=O)n2C ; Cc1oc2c(c1CC(=O)Nc1nc(-c3ccc(OC(F)(F)F)c(Cl)c3)cs1)c(=O)n(C)c(=O)n2C]

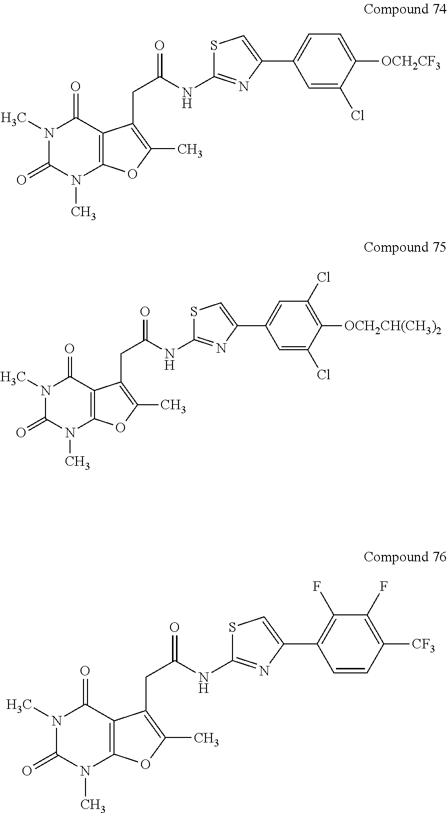

The preparation of above said compounds is described in WO2010109329.

In one aspect, TRPA1 antagonists useful in the context of the invention are selected from those compounds generically or specifically disclosed in WO2010109328. Accordingly, TRPA1 antagonists useful in the context of the invention has the formula (VI)

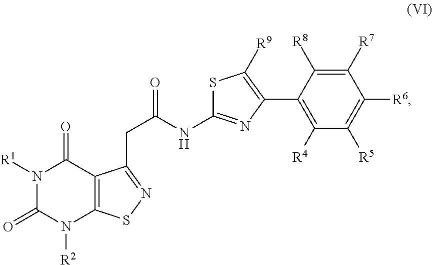

or a pharmaceutically-acceptable salt thereof.
wherein, R¹ and R², which may be the same or different, are each independently hydrogen or (C₁-C₄)alkyl; and

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists useful in the context of the invention are mentioned below:

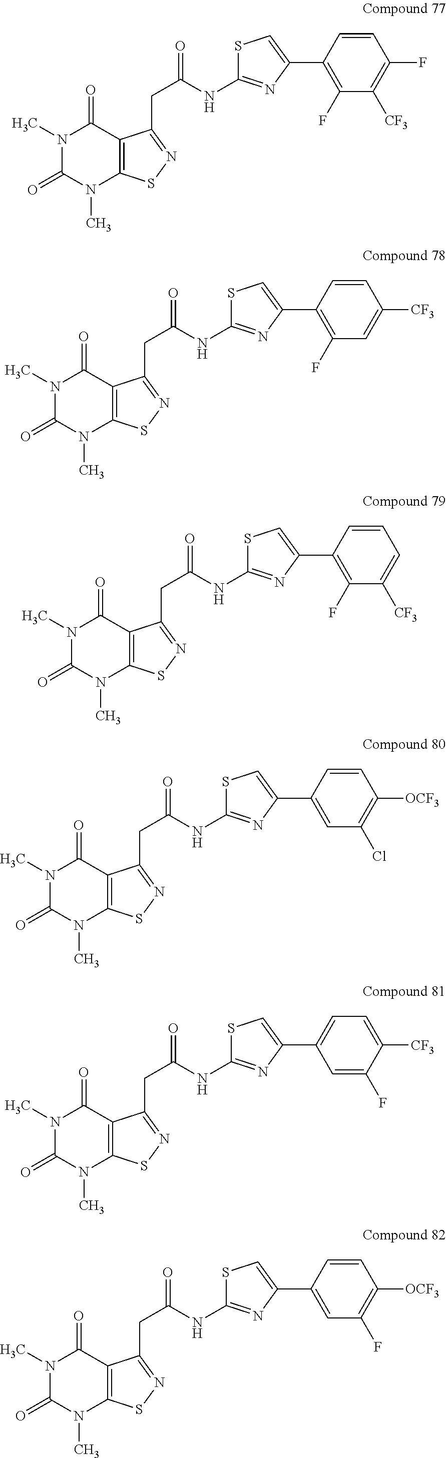 [SMILES: Cn1c(=O)c2c(CC(=O)Nc3nc(-c4ccc(F)c(C(F)(F)F)c4F)cs3)nsc2n(C)c1=O ; Cn1c(=O)c2c(CC(=O)Nc3nc(-c4ccc(C(F)(F)F)cc4F)cs3)nsc2n(C)c1=O ; Cn1c(=O)c2c(CC(=O)Nc3nc(-c4cccc(C(F)(F)F)c4F)cs3)nsc2n(C)c1=O ; Cn1c(=O)c2c(CC(=O)Nc3nc(-c4ccc(OC(F)(F)F)c(Cl)c4)cs3)nsc2n(C)c1=O ; Cn1c(=O)c2c(CC(=O)Nc3nc(-c4ccc(C(F)(F)F)c(F)c4)cs3)nsc2n(C)c1=O ; Cn1c(=O)c2c(CC(=O)Nc3nc(-c4ccc(OC(F)(F)F)c(F)c4)cs3)nsc2n(C)c1=O]

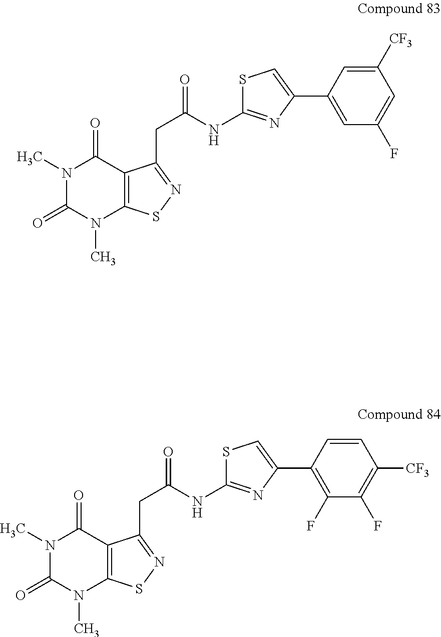 [SMILES: Cn1c(=O)c2c(CC(=O)Nc3nc(-c4cc(F)cc(C(F)(F)F)c4)cs3)nsc2n(C)c1=O ; Cn1c(=O)c2c(CC(=O)Nc3nc(-c4ccc(C(F)(F)F)c(F)c4F)cs3)nsc2n(C)c1=O]

The preparation of above said compounds is described in WO2010109328.

In one aspect, TRPA1 antagonists useful in the context of the invention are selected from those compounds generically or specifically disclosed in WO2010125469. Accordingly, TRPA1 antagonists useful in the context of the invention have the formulas (VIIa, VIIb and VIIc):

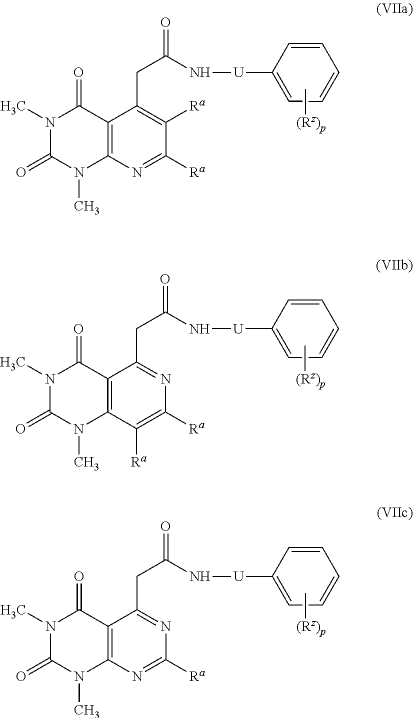

or pharmaceutically acceptable salt thereof,
wherein,

at each occurrence, R^{a} is selected from hydrogen, cyano, halogen, substituted or unsubstituted alkyl, haloalkyl, alkenyl, alkynyl, alkoxy, cycloalkyl and cycloalkylalkyl;

U is substituted or unsubstituted five membered heterocycle, for example selected from the group consisting of

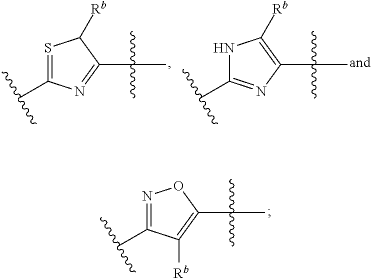

at each occurrence, R^{b} is independently selected from hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

at each occurrence, R^{z} is independently selected from halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring, heterocyclylalkyl, COOR^{x}, CONR^{x}R^{y}, S(O)ₘNR^{x}R^{y}, NR^{x}(CR^{x}R^{y})ₙOR^{x}, (CH₂)ₙNR^{x}R^{y}, NR^{x}(CR^{x}R^{y})ₙCONR^{x}R^{y}, (CH₂)ₙNHCOR^{x}, (CH₂)ₙNH(CH₂)ₙSO₂R^{x} and (CH₂)ₙNHSO₂R^{x};

at each occurrence, R^{x} and R^{y} are independently selected from hydrogen, hydroxyl, halogen, substituted or unsubstituted alkyl, alkenyl, alkynyl, cycloalkyl, cycloalkylalkyl, cycloalkenyl, aryl, arylalkyl, heteroaryl, heteroarylalkyl, heterocyclic ring and heterocyclylalkyl;

at each occurrence, ‘m’ and ‘n’ are independently selected from 0 to 2, both inclusive; and ‘p’ is independently selected from 0 to 5, both inclusive.

Few representative TRPA1 antagonists useful in the context of the invention are mentioned below:

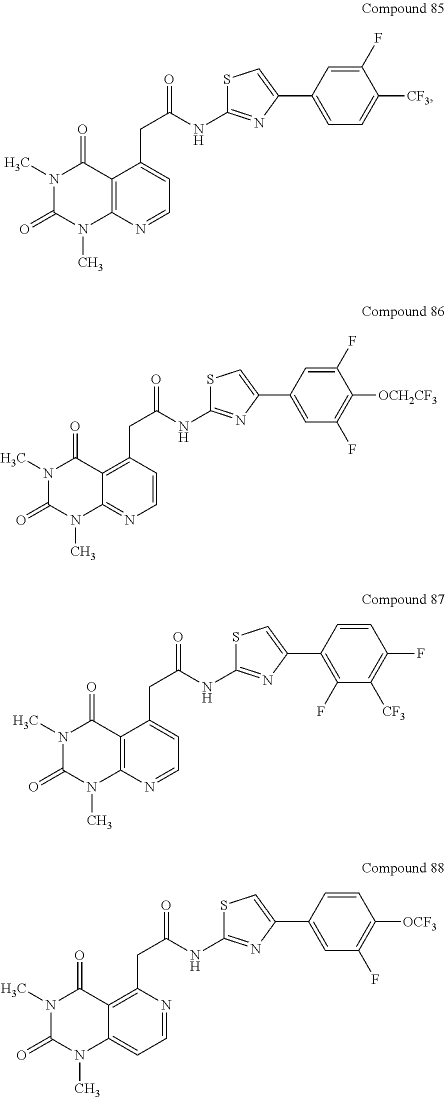

The preparation of above said compounds is described in WO2010125469.

In one aspect, the TRPA1 antagonist useful in the context of the invention is Compound 89:

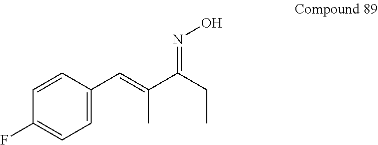

In one embodiment, the TRPA1 antagonist useful in the context of the invention is Compound 90:

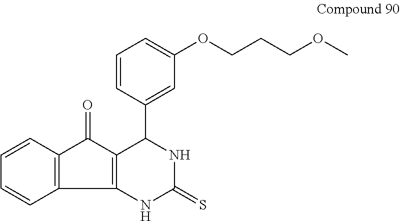 [SMILES: COCCCOc1cccc(C2NC(=S)NC3=C2C(=O)c2ccccc23)c1]

In an embodiment, TRPA1 antagonists useful in the context of the invention has the formula

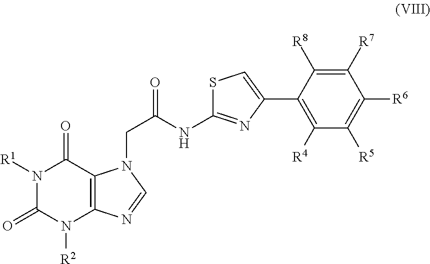

or a pharmaceutically-acceptable salt thereof
wherein,
R¹, R² and R^{a}, which may be the same or different, are each independently hydrogen or (C₁-C₄)alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

A representative TRPA1 antagonist useful in the context of the invention is Compound 91:

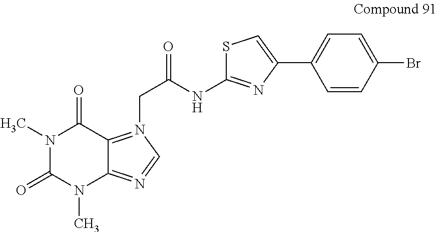

The Compound 91 may be prepared, for example, by following the process provided for the preparation of similar compounds in PCT publication No. WO2007073505.

In another aspect, TRPA1 antagonists useful in the context of the invention are selected from those compounds generically or specifically disclosed in WO2011114184. Accordingly, a TRPA1 antagonist useful in the context of the invention has the formula (IX):

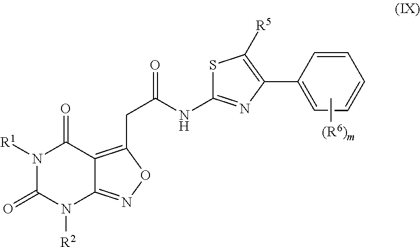

or a pharmaceutically-acceptable salt thereof

wherein at each occurrence, R¹ and R² are independently selected from hydrogen or substituted or unsubstituted alkyl;

at each occurrence, R⁵ is selected from hydrogen, halogen or substituted or unsubstituted alkyl;

at each occurrence, R⁶ is selected from hydrogen, cyano, nitro, halogen, hydroxyl, substituted or unsubstituted alkyl, alkenyl, alkynyl, cycloalkyl, cycloalkylalkyl, cycloalkenyl, haloalkyl, haloalkoxy, cycloalkylalkoxy, aryl, arylalkyl, biaryl, heteroaryl, heteroarylalkyl, heterocyclic ring and heterocyclylalkyl.

A representative TRPA1 antagonist useful in the methods of the invention is mentioned below:

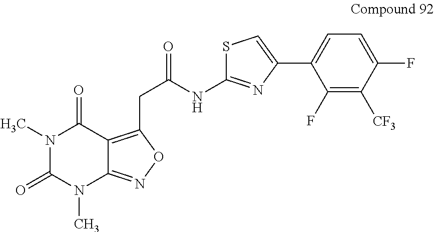 [SMILES: Cn1c(=O)c2c(CC(=O)Nc3nc(-c4ccc(F)c(C(F)(F)F)c4F)cs3)onc2n(C)c1=O]

The preparation of above said compounds is described in WO2011114184.

In another aspect, TRPA1 antagonist useful in the context of the invention has the formula (X):

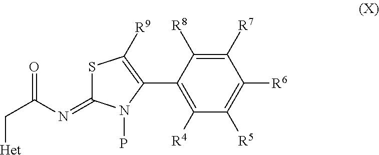

wherein, ‘Het’ is selected from groups consisting of

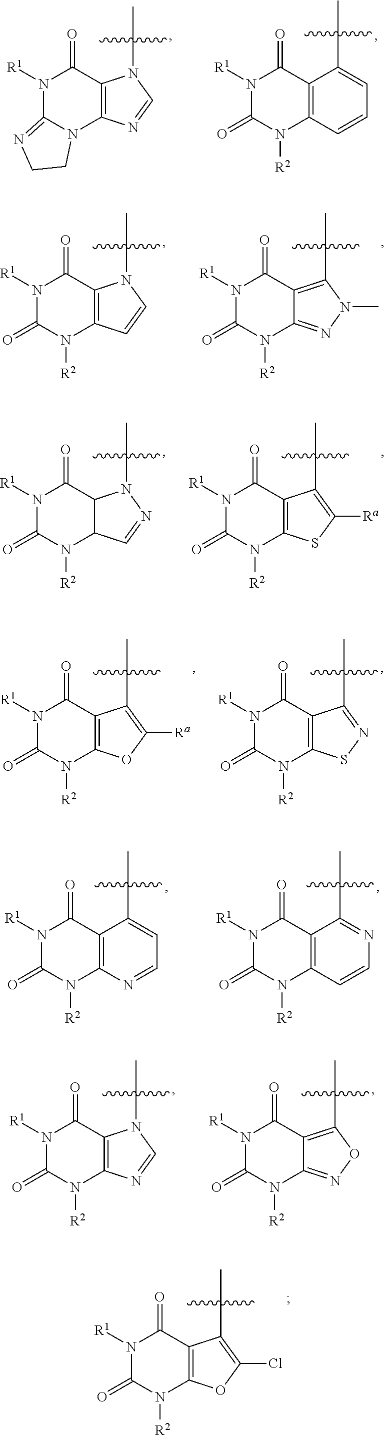

P is selected from

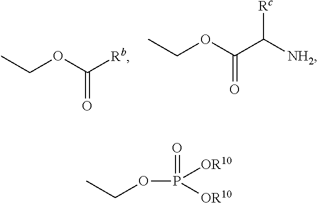

R¹, R² and R^{a}, which may be the same or different, are each independently hydrogen or (C₁-C₄) alkyl;

R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;

R^{b} and R^{c} independently selected from hydrogen, substituted or unsubstituted alkyl arylalkyl, amino acid and heterocyclic ring;

R¹⁰ is selected from hydrogen, alkyl, arylalkyl and pharmaceutically acceptable cation.

Few representative TRPA1 antagonists useful in the context of the invention are mentioned below:

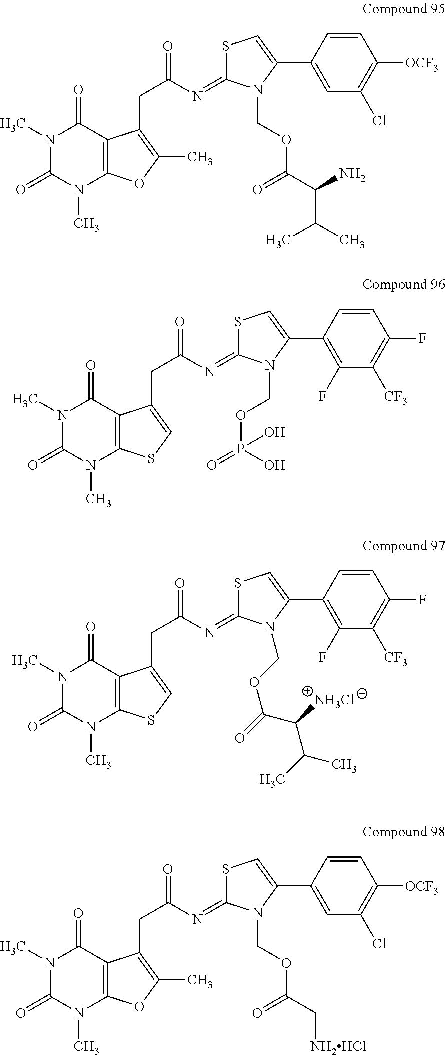

In another aspect, TRPA1 antagonists useful in the context of the invention are selected from those compounds generically or specifically disclosed in WO2011114184. Accordingly, TRPA1 antagonist useful in the context of the invention has the formula (XI):

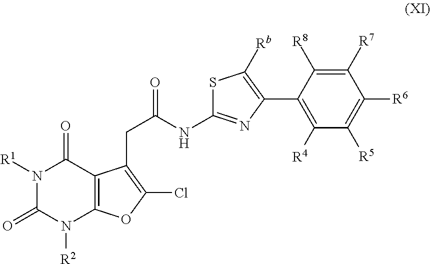

or a pharmaceutically acceptable salt thereof,

wherein, R¹, and R² are independently hydrogen or (C₁-C₄)alkyl; and

R⁴, R⁵, R⁶, R⁷, R⁸ and R⁹, which may be same or different, are each independently selected from halogen haloalkyl, dialkylamino, and haloalkoxy.

Few representative TRPA1 antagonists useful in the context of the invention are mentioned below:

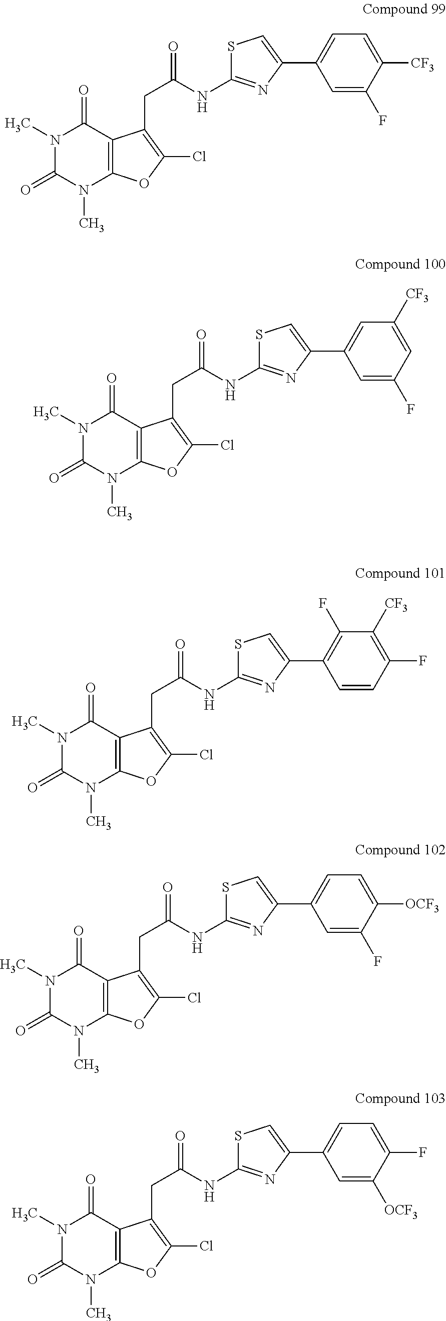 [SMILES: Cn1c(=O)c2c(CC(=O)Nc3nc(-c4ccc(C(F)(F)F)c(F)c4)cs3)c(Cl)oc2n(C)c1=O ; Cn1c(=O)c2c(CC(=O)Nc3nc(-c4cc(F)cc(C(F)(F)F)c4)cs3)c(Cl)oc2n(C)c1=O ; Cn1c(=O)c2c(CC(=O)Nc3nc(-c4ccc(F)c(C(F)(F)F)c4F)cs3)c(Cl)oc2n(C)c1=O ; Cn1c(=O)c2c(CC(=O)Nc3nc(-c4ccc(OC(F)(F)F)c(F)c4)cs3)c(Cl)oc2n(C)c1=O ; Cn1c(=O)c2c(CC(=O)Nc3nc(-c4ccc(F)c(OC(F)(F)F)c4)cs3)c(Cl)oc2n(C)c1=O]

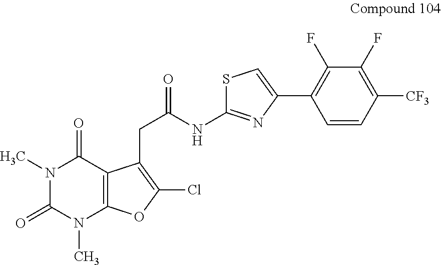 [SMILES: Cn1c(=O)c2c(CC(=O)Nc3nc(-c4ccc(C(F)(F)F)c(F)c4F)cs3)c(Cl)oc2n(C)c1=O]

The preparation of above said compounds is described in WO2011114184.

In an aspect, TRPA1 antagonists useful in the context of the invention, is selected from one of the following formulae: (XII) or (D)

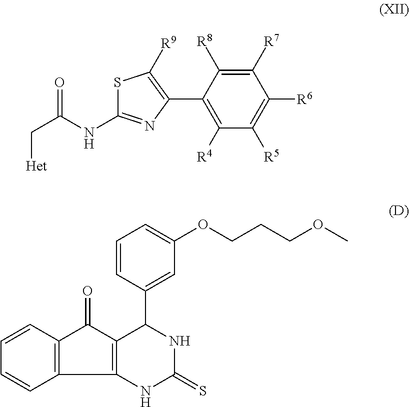

or a pharmaceutically-acceptable salt thereof, wherein, ‘Het’ is selected from the group consisting of

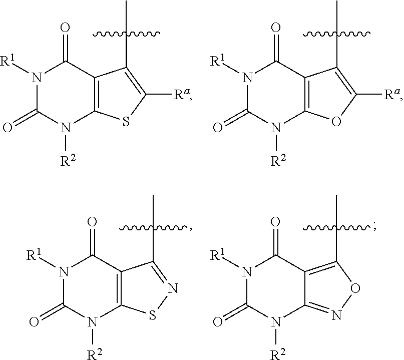

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

Few representative TRPA1 antagonists of the formula (XII) useful in the context of the invention are compound 52, compound 73 and compound 84 as described above.

The glucocorticoid, as contemplated herein, including prednisolone, beclomethasone, dexamethasone, fluticasone, mometasone, triamcinolone, prednisone, methylprednisolone, budesonide, ciclesonide, and flunisolide or their salt may be present in the form of their isomers, polymorphs, and solvates, including hydrates, all of which are included in the scope of the invention. Preferably, the glucocorticoid includes fluticasone, prednisolone, budesonide or salts thereof.

In an embodiment, the present invention relates to a pharmaceutical composition comprising synergistically effective amount of a TRPA1 antagonist having an IC₅₀ for inhibiting human TRPA1 receptor activity of less than 1 micromolar, and a glucocorticoid. Preferably, the TRPA1 antagonist of the present invention has an IC₅₀ for inhibiting human TRPA1 receptor activity of less than 500 nanomolar, or more preferably less than 250 nanomolar, as measured by a method described herein.

In another embodiment, the present invention relates to a pharmaceutical composition comprising synergistically effective amount of a TRPA1 antagonist having an IC₅₀ for inhibiting human TRPA1 receptor activity of less than 1 micromolar having structure of formulae: (XII) or (D)

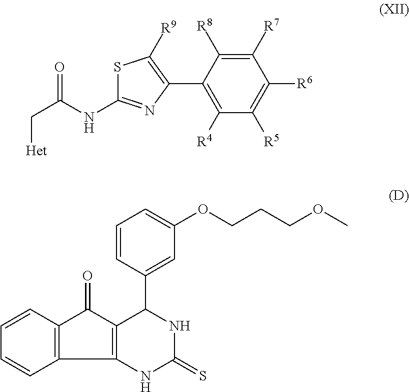

or a pharmaceutically-acceptable salt thereof, wherein, ‘Het’ is selected from the group consisting of

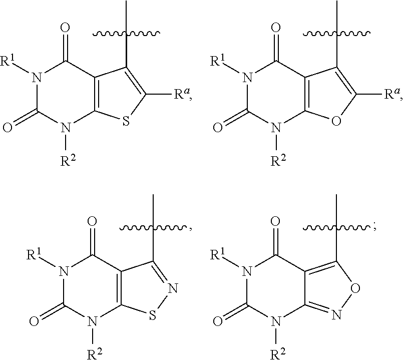

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl and a glucocorticoid.

In yet another embodiment, the present invention relates to a pharmaceutical composition comprising synergistically effective amount of a TRPA1 antagonist having structure of formula:

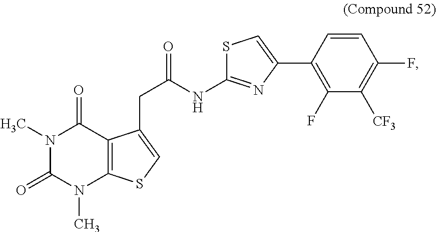 [SMILES: Cn1c(=O)c2c(CC(=O)Nc3nc(-c4ccc(F)c(C(F)(F)F)c4F)cs3)csc2n(C)c1=O]

and a glucocorticoid.

In another embodiment, there is provided a pharmaceutical composition comprising synergistically effective amount of a TRPA1 antagonist having an IC₅₀ for inhibiting human TRPA1 receptor activity of less than 1 micromolar and a glucocorticoid in a weight ratio ranging from about 1:0.001 to about 1:5000.

In an embodiment, the present invention relates to a pharmaceutical composition comprising synergistically effective amount of a TRPA1 antagonist having structure of formula:

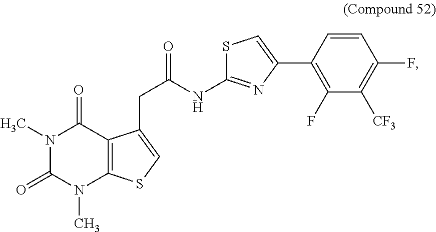 [SMILES: Cn1c(=O)c2c(CC(=O)Nc3nc(-c4ccc(F)c(C(F)(F)F)c4F)cs3)csc2n(C)c1=O]

and a glucocorticoid selected from a group consisting of prednisolone, beclomethasone, dexamethasone, fluticasone, mometasone, triamcinolone, prednisone, methylprednisolone, budesonide, ciclesonide, flunisolide or salts thereof. In an aspect of this embodiment, the pharmaceutical composition is a fixed dose combination.

In another aspect of this embodiment, the composition is for oral administration and the TRPA1 antagonist and the glucocorticoid selected from the group consisting of prednisolone, budesonide or salts thereof are present in a weight ratio ranging from about 1:0.001 to about 1:100. In an aspect of the embodiment, the TRPA1 antagonist and the glucocorticoid are present in a weight ratio ranging from about 1:0.003 to about 1:15. The glucocorticoid for oral administration includes prednisolone, budesonide or salts thereof. The TRPA1 antagonist and the glucocorticoid are present in a weight ratio of about 1:0.001; 1:0.003; 1:0.001; 1:0.01; 1:0.1; 1:0.13; 1:0.15; 1:0.2; 1:0.3; 1:0.5; 1:0.6; 1:0.75; 1:1; 1:2; 1:3; 1:4; 1:5; 1:7.5; 1:10; 1:12; 1:15; 1:18; 1:20; 1:25; 1:30; 1:40; 1:50; 1:75 or 1:100.

In yet another aspect of this embodiment, the composition is for inhalation administration and the TRPA1 antagonist and the glucocorticoid selected from the group consisting of fluticasone, prednisolone, budesonide or salts thereof are present in a weight ratio ranging from about 1:0.001 to about 1:5000. In an aspect of the embodiment, the TRPA1 antagonist and the glucocorticoid are present in a weight ratio ranging from about 1:0.0025 to about 1:3200. The glucocorticoid for inhalation administration includes fluticaone, prednisolone, budesonide or salts thereof. The TRPA1 antagonist and the glucocorticoid are present in a weight ratio of about 1:0.001; 1:0.025; 1:0.003; 1:0.005; 1:0.001; 1:0.01; 1:0.1; 1:0.2; 1:0.3; 1:0.5; 1:0.6; 1:0.75; 1:1; 1:2; 1:3; 1:4; 1:5; 1:7.5; 1:10; 1:12; 1:15; 1:18; 1:20; 1:25; 1:30; 1:40; 1:50; 1:75; 1:100; 1:200; 1:500; 1:750; 1:1000; 1:1500; 1:2000; 1:2500; 1:3000; 1:3200; 1:3500; 1:4000 or 1:5000.

As contemplated herein, the active ingredients may be administered together in a single dosage form or they may be administered in different dosage forms. They may be administered at the same time or they may be administered either close in time or remotely, such as, where one drug is administered in the morning and the second drug is administered in the evening. The combination may be used prophylactically or after the onset of symptoms has occurred.

In a preferred embodiment, both the active ingredients i.e., TRPA1 antagonist and the glucocorticoid are formulated as a pharmaceutical composition suitable for administration by the same route (e.g., both the actives by oral or inhalation route), or by different routes (e.g., one active by oral and the other active by inhalation route).

The pharmaceutical compositions for oral administration may be in conventional forms, for example, tablets, capsules, granules (synonymously, “beads” or “particles” or “pellets”), suspensions, emulsions, powders, dry syrups, and the like. The capsules may contain granule/pellet/particle/mWini-tablets/mini-capsules containing the active ingredients. The amount of the active agent that may be incorporated in the pharmaceutical composition may range from about 1% w/w to about 98% w/w or from about 5% w/w to about 90% w/w.

The pharmaceutical compositions for parenteral administration include but are not limited to solutions/suspension/emulsion for intravenous, subcutaneous or intramuscular injection/infusion, and implants. The pharmaceutical compositions for transdermal or transmucosal administration include but are not limited to patches, gels, creams, ointments and the like.

As set forth above, the pharmaceutical composition includes at least one pharmaceutically acceptable excipient, which includes but is not limited to one or more of the following; diluents, glidants and lubricants, preservatives, buffering agents, chelating agents, polymers, gelling agents/viscosifying agents, surfactants, solvents and the like.

In an embodiment, the present invention provides a process for the preparing a pharmaceutical composition comprising TRPA1 antagonist and a glucocorticoid and a pharmaceutically acceptable excipient, wherein the composition is in the form of a fixed dose combination formulation. The process comprises admixing TRPA1 antagonist with the glucocorticoid. Alternately, the process comprises formulating TRPA1 antagonist and the glucocorticoid in such a way that they are not in intimate contact with each other.

In another embodiment, the invention relates to a process for preparing a pharmaceutical composition comprising TRPA1 antagonist, a glucocorticoid and a pharmaceutically acceptable excipient, wherein the composition is in the form of kit comprising separate formulations of TRPA1 antagonist and the glucocorticoid.

The process for making the pharmaceutical composition may for example include, (1) granulating either or both the active ingredients, combined or separately, along with pharmaceutically acceptable carriers so as to obtain granulate, and (2) converting the granulate into suitable dosage forms for oral administration. The typical processes involved in the preparation of the pharmaceutical combinations include various unit operations such as mixing, sifting, solubilizing, dispersing, granulating, lubricating, compressing, coating, and the like. These processes, as contemplated by a person skilled in the formulation art, have been incorporated herein for preparing the pharmaceutical composition of the present invention.

### Methods of Treatment

Asthma and COPD are major chronic diseases related to airway obstruction. The Global Initiative for Chronic Obstructive Lung Disease provides guidelines for the distinction between asthma and COPD. Asthma is believed to be a chronic inflammatory disease wherein the airflow limitation is more or less reversible while it is more or less irreversible in case of COPD. Asthma among other things is believed to be triggered by inhalation of sensitizing agents (like allergens) unlike noxious agents (like particles and certain gases) in case of COPD. Though both are believed to have an inflammatory component, the inflammation in asthma is believed to be mostly eosinophilic and CD-4 driven, while it is believed to be mostly neutrophilic and CD-8 driven in COPD.

Asthma is characterized by chronic airway inflammation and airway hyper-responsiveness (AHR). Klein et al. (Pulmonary Pharmacology and Therapeutics, 2008; 21, 648-656 disclose that airway eosinophilia was found to correlate with asthma severity and AHR in both atopic and non-atopic asthma patients.

Asthma is clinically classified according to the frequency of symptoms, forced expiratory volume in 1 second (FEV₁), peak expiratory flow rate and severity (e.g., acute, intermittent, mild persistent, moderate persistent, and severe persistent). Asthma may also be classified as allergic (extrinsic) or non-allergic (intrinsic), based on whether symptoms are precipitated by allergens or not. Asthma can also be categorized according to following types viz., nocturnal asthma, bronchial asthma, exercise induced asthma, occupational asthma, seasonal asthma, silent asthma, and cough variant asthma.

It is believed that reduction of eosinophil count and increase in FEV 1 are important components of the treatment of respiratory disorders such as asthma. Ulrik CS, 1995 (Peripheral eosinophil counts as a marker of disease activity in intrinsic and extrinsic asthma; Clinical and Experimental Allergy; 1995, Volume 25, pages 820-827) discloses the relationship between eosinophil count and severity of asthmatic symptoms. It describes that in childhood and adulthood subjects, there exists an inverse correlation between number of eosinophils and FEV1% (r=−0.75, P<0.001, and r=−0.80, P<0.001, respectively).

COPD, also known as chronic obstructive lung disease (COLD), chronic obstructive airway disease (COAD), or chronic obstructive respiratory disease (CORD), is believed to be the co-occurrence of chronic bronchitis (characterized by a long-term cough with mucus) and emphysema (characterized by destruction of the lungs over time), a pair of commonly co-existing diseases of the lungs in which the airways become narrowed. This leads to a limitation of the flow of air to and from the lungs, causing shortness of breath. An acute exacerbation of COPD is a sudden worsening of COPD symptoms (shortness of breath, quantity and color of phlegm) that typically lasts for several days and is believed to be triggered by an infection with bacteria or viruses or by environmental pollutants. Based on the FEV₁ values, COPD can be classified as mild, moderate, severe and very severe.

Various classes of drugs are currently being used for the treatment and/or prophylaxis of respiratory disorders like asthma and COPD. Some of the classes of such drugs are leukotriene receptor antagonists, antihistamines, beta-2 agonists, anticholinergic agents and corticosteroids.

In an embodiment, the present invention relates to a method of treating a respiratory disorder in a subject in need thereof, said method comprising administering to the subject the pharmaceutical composition comprising synergistically effective amount of a TRPA1 antagonist having an IC₅₀ for inhibiting human TRPA1 receptor activity of less than 1 micromolar and a glucocorticoid. In an aspect of this embodiment, the TRPA1 antagonist has an IC₅₀ for inhibiting human TRPA1 receptor activity of less than 1 micromolar having structure of formulae: (XII) or (D)

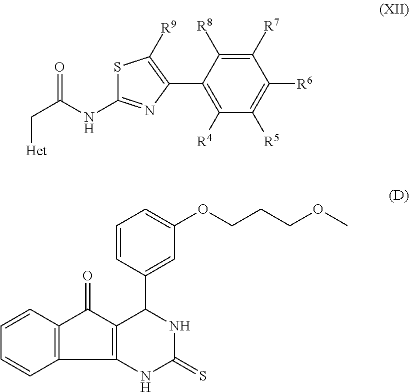

or a pharmaceutically-acceptable salt thereof, wherein, ‘Het’ is selected from the group consisting of

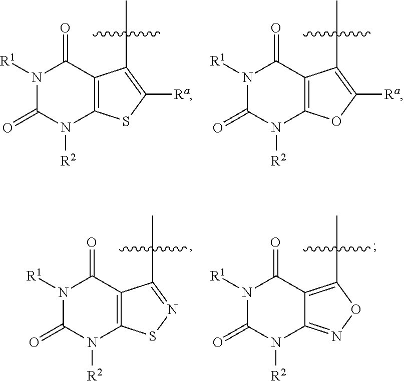

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In a further embodiment, the present invention relates to a method of treating a respiratory disorder in a subject in need thereof, said method comprising administering the subject a pharmaceutical composition comprising synergistically effective amount of a TRPA1 antagonist having an IC₅₀ for inhibiting human TRPA1 receptor activity of less than 1 micromolar and glucocorticoid selected from a group consisting of prednisolone, beclomethasone, dexamethasone, fluticasone, mometasone, triamcinolone, prednisone, methylprednisolone, budesonide, ciclesonide, flunisolide or salts thereof. In an aspect of the embodiment, the glucocorticoid is fluticasone, prednisolone, budesonide or salts thereof.

In a further embodiment, the present invention relates to use of synergistically effective amount of a TRPA1 antagonist having an IC₅₀ for inhibiting human TRPA1 receptor activity of less than 1 micromolar and a glucocorticoid in the preparation of a pharmaceutical composition of the present invention for the treatment of a respiratory disorder in a subject in need thereof. In an aspect of this embodiment, the TRPA1 antagonist has an IC₅₀ for inhibiting human TRPA1 receptor activity of less than 1 micromolar having structure of formulae: (XII) or (D)

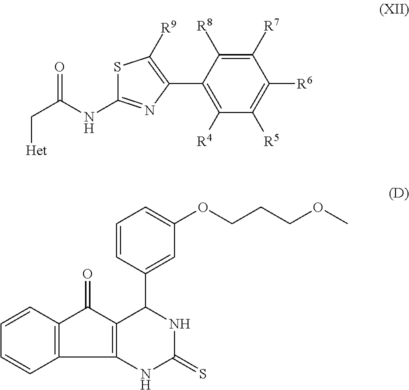

or a pharmaceutically-acceptable salt thereof, wherein, ‘Het’ is selected from the group consisting of

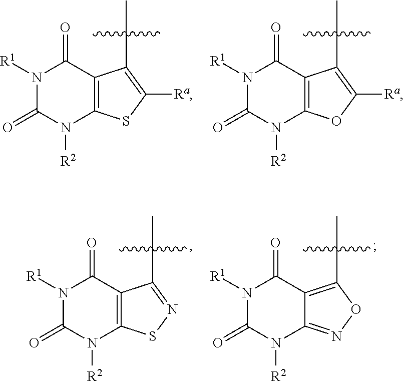

R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl.

In a further embodiment, the present invention relates to a pharmaceutical composition comprising synergistically effective amount of a TRPA1 antagonist having an IC₅₀ for inhibiting human TRPA1 receptor activity of less than 1 micromolar and a glucocorticoid for the treatment of a respiratory disorder in a subject in need thereof.

In an embodiment, the present invention relates to a method of treating a respiratory disorder in a subject in need thereof, said method comprising administering to the subject the pharmaceutical composition comprising synergistically effective amount of a TRPA1 antagonist having structure of formula:

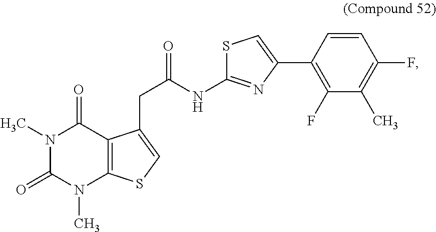 [SMILES: Cc1c(F)ccc(-c2csc(NC(=O)Cc3csc4c3c(=O)n(C)c(=O)n4C)n2)c1F]

and a glucocorticoid. In an aspect of this embodiment, the glucocorticoid is selected from a group consisting of prednisolone, beclomethasone, dexamethasone, fluticasone, mometasone, triamcinolone, prednisone, methylprednisolone, budesonide, ciclesonide, flunisolide or salts thereof.

In an embodiment, the present invention relates to a method of treating a respiratory disorder by reducing eosinophils count and/or increasing FEV1 value in a subject in need thereof, said method comprising administering to the subject the pharmaceutical composition comprising synergistically effective amount of a TRPA1 antagonist having structure of formula:

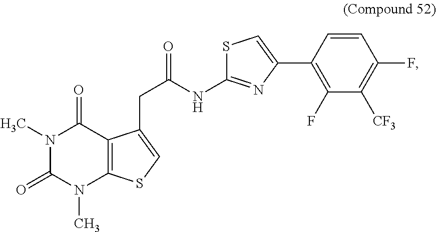 [SMILES: Cn1c(=O)c2c(CC(=O)Nc3nc(-c4ccc(F)c(C(F)(F)F)c4F)cs3)csc2n(C)c1=O]

and a glucocorticoid, thereby reducing said eosinophil count and/or increasing FEV1 value in said subject. In an aspect of this embodiment, the glucocorticoid is selected from a group consisting of prednisolone, beclomethasone, dexamethasone, fluticasone, mometasone, triamcinolone, prednisone, methylprednisolone, budesonide, ciclesonide, flunisolide or salts thereof. In another aspect of this embodiment, the respiratory disorder is asthma.

In an embodiment, the present invention relates to a method of treating a respiratory disorder by reducing airway inflammation in a subject in need thereof, said method comprising administering to the subject the pharmaceutical composition comprising synergistically effective amount of a TRPA1 antagonist having structure of formula:

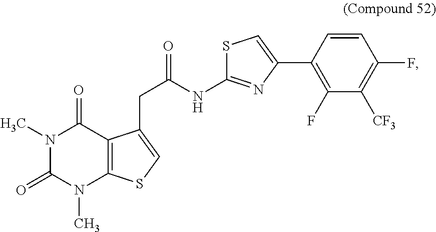 [SMILES: Cn1c(=O)c2c(CC(=O)Nc3nc(-c4ccc(F)c(C(F)(F)F)c4F)cs3)csc2n(C)c1=O]

and a glucocorticoid, thereby reducing said airway inflammation.

In an embodiment, the present invention relates to a method of reducing eosinophils count and/or increasing FEV1 value in a subject in need thereof, said method comprising administering to the subject the pharmaceutical composition comprising synergistically effective amount of a TRPA1 antagonist having structure of formula:

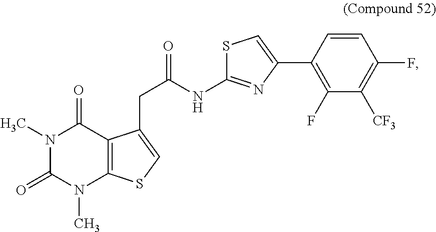 [SMILES: Cn1c(=O)c2c(CC(=O)Nc3nc(-c4ccc(F)c(C(F)(F)F)c4F)cs3)csc2n(C)c1=O]

and a glucocorticoid, thereby reducing said eosinophil count and/or increasing FEV1 value in said subject. In an aspect of this embodiment, the glucocorticoid is selected from a group consisting of prednisolone, beclomethasone, dexamethasone, fluticasone, mometasone, triamcinolone, prednisone, methylprednisolone, budesonide, ciclesonide, flunisolide or salts thereof.

In an embodiment, the present invention relates to a method of reducing airway inflammation in a subject in need thereof, said method comprising administering to the subject the pharmaceutical composition comprising synergistically effective amount of a TRPA1 antagonist having structure of formula:

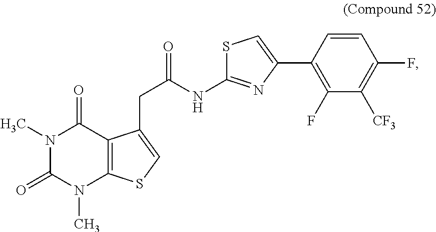 [SMILES: Cn1c(=O)c2c(CC(=O)Nc3nc(-c4ccc(F)c(C(F)(F)F)c4F)cs3)csc2n(C)c1=O]

and a glucocorticoid, thereby reducing said airway inflammation in said subject.

In another embodiment, the present invention relates to use of synergistically effective amount of a TRPA1 antagonist having structure of formula:

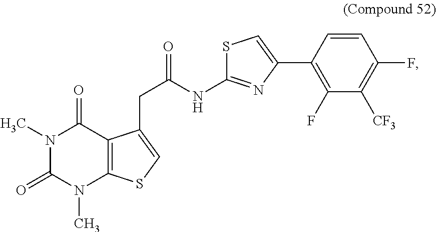 [SMILES: Cn1c(=O)c2c(CC(=O)Nc3nc(-c4ccc(F)c(C(F)(F)F)c4F)cs3)csc2n(C)c1=O]

and glucocorticoids in the preparation of a pharmaceutical composition of the present invention for the treatment of a respiratory disorder in a subject in need thereof. In an aspect of this embodiment, the glucocorticoid is selected from a group consisting of prednisolone, beclomethasone, dexamethasone, fluticasone, mometasone, triamcinolone, prednisone, methylprednisolone, budesonide, ciclesonide, flunisolide or salts thereof.

In a further embodiment, the present invention relates to a pharmaceutical composition comprising synergistically effective amount of a TRPA1 antagonist having structure of formula:

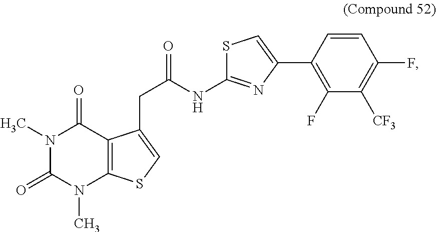 [SMILES: Cn1c(=O)c2c(CC(=O)Nc3nc(-c4ccc(F)c(C(F)(F)F)c4F)cs3)csc2n(C)c1=O]

and a glucocorticoid for the treatment of a respiratory disorder in a subject in need thereof.

The therapeutically effective amount of TRPA1 antagonist to be administered per day ranges from about 10 μg/kg to about 20 mg/kg, and preferably from about 50 μg/kg to about 15 mg/kg.

The therapeutically effective amount of fluticasone or its salt to be administered per day ranges from about 10 μg to about 5 mg, and preferably from about 50 μg to about 3 mg, and more preferably from about 100 μg to about 2 mg. Preferably, the discrete dosage strengths of fluticasone or its salt to be administered per day are 50 μg; 100 μg and 250 μg.

The therapeutically effective amount of prednisolone or its salt to be administered per day ranges from about 1 mg to about 100 mg; and preferably from about 2 mg to about 75 mg; and more preferably from about 5 mg to about 60 mg. Preferably, the discrete dosage strengths of prednisolone or its salt to be administered per day are 5 mg and 15 mg.

The therapeutically effective amount of budesonide or its salt to be administered per day ranges from about 0.01 mg to about 20 mg; and preferably from about 0.05 mg to about 10 mg; and more preferably from about 0.09 mg to about 9 mg. Preferably, the discrete dosage strengths of budesonide or its salt to be administered per day are 80 μg and 160 μg.

The optimal dose of the active ingredient or the combination of the active ingredients can vary as a function of the severity of disease, route of administration, composition type, the patient body weight, the age and the general state of mind of the patient, and the response to behavior to the active ingredient or the combination of the active ingredients.

In the pharmaceutical composition as described herein, the active ingredient may be in the form of a single dosage form (i.e., fixed-dose formulation in which both the active ingredients are present together) or they may be divided doses, formulated separately, each in its individual dosage forms but as part of the same therapeutic treatment, program or regimen, either once daily or two/three/four times a day.

Alternately, the invention relates to a pharmaceutical composition wherein the composition is in the form of kit comprising separate formulations of TRPA1 antagonist and the glucocorticoid. The separate formulations are to be administered by same or different routes, either separately, simultaneously, or sequentially, where the sequential administration is close in time or remote in time. For sequential administration, the period of time may be in the range from 10 min to 12 hours.

Various animal models have been used for the evaluation of the therapeutic efficacy of drug candidates for respiratory disorders like asthma and COPD. For example, commonly used strategy for evaluation of drug candidates in asthma is the allergen sensitization and challenge method. The commonly used such model is the ovalbumin (OVA) sensitization and challenge in laboratory animals. Another model that can be used is the methacholine challenge test by using invasive whole body plethysmograph.

A commonly used model for evaluation of drug candidates in COPD involves the chronic exposure of the animal to SO₂ or tobacco/cigarette smoke. The model is believed to generate sloughing of epithelial cells, increase in the mucus secretions, increase in the polymorphonuclear cells and pulmonary resistance, and increase in the airway hyper-responsiveness (in rats). Another model that can be used for evaluation of drug candidates in COPD involves the exposure of animals (e.g., rats) to lipopolysaccharide (LPS). The exposure to LPS is believed to result in the influx of neutrophils in the lungs, a condition that is believed to be one of the characteristics of COPD.

It will be understood that various modifications may be made to the embodiments disclosed herein. Therefore the above description should not be construed as limiting, but merely as exemplifications of preferred embodiments. Other arrangements and methods may be implemented by those skilled in the art without departing from the scope and spirit of this invention.

The following examples are provided to enable one skilled in the art to practice the invention and are merely illustrative of the invention. The examples should not be read as limiting the scope of the invention.

### EXAMPLES

### Example 1

### Determination of IC₅₀ of TRPA1 Antagonists

The human IC₅₀ values were measured by the following method: The inhibition of TRPA1 receptor activation is measured as inhibition of allylisothiocyanate (AITC) induced cellular uptake of radioactive calcium. Test compound solution is prepared in a suitable solvent. Human TRPA1 expressing CHO cells are grown in suitable medium. Cells are treated with test compounds followed by addition of AITC. Cells are washed, lysed and the radioactivity in the lysate is measured in Packard Top count after addition of liquid scintillant.

The concentration response curves for compounds are plotted as a % of maximal response obtained in the absence of test antagonist, and the IC₅₀ values are calculated from such concentration response curve by nonlinear regression analysis using GraphPad PRISM software.

TABLE 1
TRPA1 antagonists having a human IC₅₀ for inhibiting
human TRPA1 receptor activity of less than 1 micromolar.

| Compound No | hTRPA1 IC₅₀ values |
|---|---|
| 1 | 920.9 nM |
| 2 | 381.8 nM |
| 3 | 73.35 nM |
| 4 | 98.32 nM |
| 5 | 66.28 nM |
| 6 | 97.42 nM |
| 7 | 47.37 nM |
| 8 | 55.02 nM |
| 9 | 102.5 nM |
| 10 | 46.74 nM |
| 11 | 46.27 nM |
| 12 | 51.68 nM |
| 13 | 48.21 nM |
| 14 | 60.42 nM |
| 15 | 53.57 nM |
| 16 | 58.94 nM |
| 17 | 56.02 nM |
| 18 | 13.38 nM |
| 19 | 26.13 nM |
| 20 | 20.09 nM |
| 21 | 48.18 nM |
| 22 | 79.77 nM |
| 23 | 43.93 nM |
| 24 | 138.1 nM |
| 25 | 58.55 nM |
| 26 | 47.91 nM |
| 27 | 65.45 nM |
| 28 | 6.49 nM |
| 29 | 11.38 nM |
| 30 | 34.03 nM |
| 31 | 17.3 nM |
| 32 | 5.96 nM |
| 33 | 5.37 nM |
| 34 | 38.46 nM |
| 35 | 18.05 nM |
| 36 | 49.92 nM |
| 37 | 12.26 nM |
| 38 | 15.92 nM |
| 39 | 26.56 nM |
| 40 | 22.82 nM |
| 41 | 11.04 nM |
| 42 | 11.38 nM |
| 43 | 18.37 nM |
| 44 | 8.36 nM |
| 45 | 26.39 nM |
| 46 | 41.31 nM |
| 47 | 33.61 nM |
| 48 | 18.12 nM |
| 49 | 3.98 nM |
| 50 | 16.73 nM |
| 51 | 4.84 nM |
| 52 | 2.49 nM |
| 53 | 18.20 nM |
| 54 | 17.74 nM |
| 55 | 2.15 nM |
| 56 | 3.38 nM |
| 57 | 1.45 nM |
| 58 | 11.88 nM |
| 59 | 2.21 nM |
| 60 | 3.54 nM |
| 61 | 2.93 nM |
| 62 | 1.68 nM |
| 63 | 9.04 nM |
| 64 | 4.52 nM |
| 65 | 6.65 nM |
| 66 | 3.63 nM |
| 67 | 13.59 nM |
| 68 | 4.84 nM |
| 69 | 7.10 nM |
| 70 | 12.57 nM |
| 71 | 3.18 nM |
| 72 | 4.16 nM |
| 73 | 8.54 nM |
| 74 | 5.29 nM |
| 75 | 3.34 nM |
| 76 | 4.02 nM |
| 77 | 5.60 nM |
| 78 | 10.57 nM |
| 79 | 5.29 nM |
| 80 | 6.28 nM |
| 81 | 6.74 nM |
| 82 | 8.04 nM |
| 83 | 4.40 nM |
| 84 | 5.35 nM |
| 85 | 8.92 nM |
| 86 | 6.91 nM |
| 87 | 19.32 nM |
| 88 | 11.45 nM |
| 89 | 98.44 nM |
| 90 | 5.61 nM |
| 91 | 451.4 nM |
| 92 | 17.08 nM |
| 95 | 88.50 nM |
| 96 | 559.3 nM |
| 97 | 21.91 nM |
| 98 | 54.29 nM |
| 99 | 5.06 nM |
| 100 | 5.15 nM |
| 101 | 10.10 nM |
| 102 | 7.67 nM |
| 103 | 27.41 nM |
| 104 | 7.58 nM |

### Example 2

### Animal Studies for the Combination of TRPA1 Antagonist and Prednisolone

The effect of the treatments (alone and in combination) on ovalbumin induced inflammation in ovalbumin (Ova) sensitized female Balb/C mice was studied. Female Balb/C (18-20 g on day 0) mice were sensitized on day 0 and 7 with 50 μg ovalbumin and 4 mg alum given i.p. Mice were challenged with 3% aerosolized ovalbumin from Day 11-13 following sensitization. Sensitized mice were randomly assigned to different treatment groups. Test compounds were triturated with 2 drops of Tween-80 and volume was made up with 0.5% methyl cellulose (MC) solution for oral administration. Animals were administered Compound 52 orally 24 hrs before first ovalbumin challenge and 2 hrs before ovalbumin challenge from Day-11 to 13. Animals were administered Prednisolone orally 24 hrs before first ovalbumin challenge and 2 hrs before ovalbumin challenge from Day-11 to 13. The animals were divided into groups as per Table 2.

TABLE 2

| | | Route of |
|---|---|---|
| Group | Treatment | administration |
| A | Saline Control | p.o |
| B | Vehicle (p.o.) treated/Ovalbumin | |
| | sensitized/Ovalbumin challenged (Vehicle) | |
| C | Compound 52 treated/Ovalbumin sensitized/ | |
| | Ovalbumin challenged (Ova + Compound 52) | |
| D | Prednisolone treated/Ovalbumin sensitized/ | |
| | Ovalbumin challenged (Ova + Prednisolone) | |
| E | (Compound 52 + Prednisolone) treated/ | |
| | Ovalbumin sensitized/Ovalbumin | |
| | challenged (Combination) | |

Broncho Alveolar Lavage (BAL) was performed at 24 hours after challenge with ovalbumin. Animals were anesthetized with an overdose of urethane, trachea was exposed and BAL was performed 4 times using 0.3 mL PBS. All aspirates of BAL were pooled and total number of cells determined using a hemocytometer. The BAL was centrifuged, and the cell pellet was used for preparation of smears. Slides were stained with Giemsa stain and a differential cell count of 500 cells based on standard morphology was performed manually.

### Calculations:

The total number of eosinophils in each BAL sample was calculated using the formula:

$$Total No . of eosinophils ( in B A Lf ) = \frac{( Total cell count \times {10}^{5} \text{/} mL \times Percent eosinophils )}{100}$$

Percent inhibition of eosinophils was calculated using the following formula:

$$\% Inhibition of eosinophils = \frac{100 [ Avg . {eosinophils}_{( v / ova / ova )} - {eosinophils}_{( compound / ova / ova )} ]}{[ Avg . {eosinophils}_{( v / ova / ova )} - Avg . {eosinophils}_{( Saline Control )} ]}$$

TABLE 3

| | | | | | % |
|---|---|---|---|---|---|
| | | | | % In- | Inhibition |
| | | Total cell | Total | hibition | of |
| Group | | number in | eosinophils | of cell | eosinophils |
| (n) | Dose | BALf | in BALf | in BALf | in BALf |
| A (6) | — | 1.2 ± 0.2 | 0.0 ± 0.0 | — | — |
| B (9) | 10 ml/Kg | 13.4 ± 5 | 7.4 ± 0.7 | — | — |
| C (6) | 1 mg/Kg | 12.2 ± 0.5 | 5.8 ± 0.5 | 10 | 21 |
| D (6) | 1 mg/Kg | 10.8 ± 1.0* | 5.6 ± 0.7 | 21 | 24 |
| E (5) | 1 mg/Kg | 6.9 ± 0.8** | 2.6 ± 0.7** | 53 | 65 |
| | of each | | | | |
| | compound | | | | |

*p < 0.05,
**p < 0.001

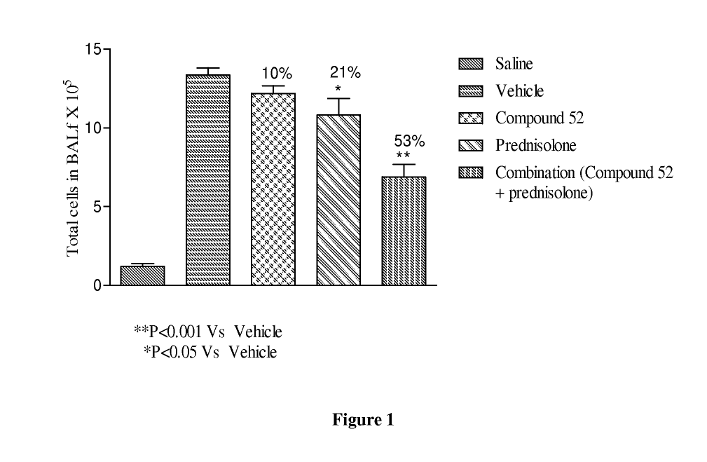

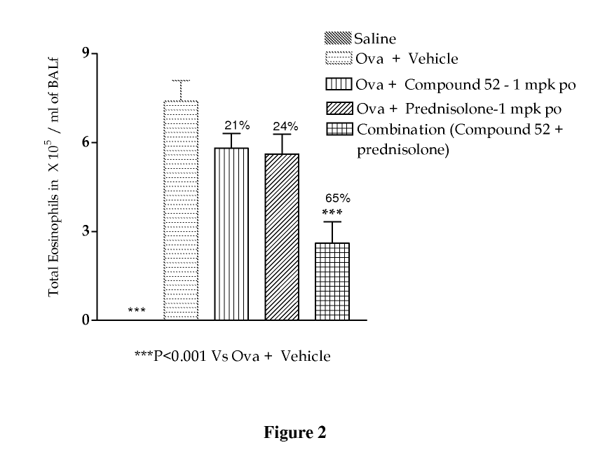

The total cell number and the total eosinophil count in the BALf was determined. It was surprisingly found that the combination of Compound 52 and prednisolone (Group E) produced significantly superior inhibition of the total cell number and eosinophils as compared to the individual activity of both treatments (Group C and Group D). The results are given in Table 3 and FIGS. 1 and 2.

### Example 3

### Animal Studies for the Combination of TRPA1 Antagonist and Fluticasone

Male Brown Norway rats (200-250 g on day 0) were sensitized subcutaneously on day 0, 14 and 21 with 0.5 ml solution containing 20 μg/ml ovalbumin and 40 mg/ml aluminium hydroxide. Simultaneously animals were injected intraperitoneally (i.p.) with 0.25 ml of B. pertussis vaccine/rat containing 4×10⁸ heat killed bacilli/ml. Rats were challenged with 1% aerosolized ovalbumin on Day 28 following sensitization.

### Animal Groupings

Animals were assigned to one of the following 5 groups during each experiment

A; Normal Saline (100 g1/animal, i.t.) and 0.5% M.C. (5 ml/kg i.p) treated/Aluminium Hydroxide Gel Sensitized/Saline Challenge-Saline Vehicle
B: Normal Saline (100 g1/animal, i.t.) and 0.5% M.C. (5 ml/kg i.p) treated/Ovalbumin challenged—Ova Vehicle
C: Fluticasone 12.5 μg/animal (i.t.) treated/Ovalbumin sensitized/Ovalbumin challenged-Fluticasone
D: Compound 52 (3 mg/kg, i.p.) treated/Ovalbumin sensitized/Ovalbumin challenged—Compound 52
E: Compound 52 (3 mg/kg, i.p)+Fluticasone (12.5 mcg/animal, i.t.) treated/Ovalbumin sensitized/Ovalbumin challenged—Combination.

TABLE 4

| | | | Ova |
|---|---|---|---|
| Group | Group Code | Dose | Challenge |
| A | Saline Vehicle | 100 μl/animal i.t. + | − |
| | | 0.5% M.C. 5 ml/kg i.p. | |
| B | Ova Vehicle | 100 μl/animal i.t. + | + |
| | | 0.5% M.C. 5 ml/kg i.p. | |
| C | Fluticasone | 100 mcg/animal i.t. | + |
| D | Compound 52 | 3 mg/kg, i.p. | + |
| E | Combination | Fluticasone (100 mcg/animal i.t.) + | + |
| | | Compound 52 (3 mg/kg, i.p.) | |

Sensitized rats were randomly assigned to different treatment groups. For i.t. administration, fluticasone was triturated and volume was made up with Normal Saline (0.9% NaCl). Compound 52 was triturated with 2 drops of Tween-80 and volume was made up with 0.5% methyl cellulose (MC) solution for i.p. administration. Animals were administered compound 52 intraperitoneally 2 hours before allergen challenge. Fluticasone was given intra-tracheally (i.t.) 24 hours and 1 hour before ovalbumin challenge. Animals were sacrificed 48 hours after ovalbumin challenge. Treated groups received the compounds intra-tracheally as mentioned in Table 4.

Broncho alveolar lavage (BAL) was performed at approximately 48 hours after ovalbumin challenge. Animals were euthanized with an overdose of urethane, trachea was exposed and BAL was performed 5 times using 2 ml PBS. All aspirates of BAL were pooled and total number of cells determined using a hemocytometer. BALf was centrifuged. The cell pellet collected after centrifugation was resuspended in 50 μL serum and used for preparation of smears.

For cell differentials, slides were stained with Leishman's stain and a differential cell count of 500 cells based on standard morphology was performed manually.

The total number of eosinophils in each BAL sample was calculated using the formula:

$$Total No . of eosinophils ( in B A L ) = \frac{Total cell count \times {10}^{5} \text{/} mL \times Percent eosinophils}{100}$$

Percent inhibition of eosinophils was calculated using the following formula:

$$\% Inhibition of eosinophils = \frac{Avg . {eosinophils}_{( Ova + Veh )} - {eosinophils}_{( treatment )}}{Avg . {eosinophils}_{( Ova + Veh )} - Avg . {eosinophils}_{( Saline + Veh )}} \times 100$$

Statistical analysis was performed using One Way ANOVA followed by Dunnett's multiple comparisons with the help of Graph Pad Prism software. Statistical significance was set at p<0.05.

### Results

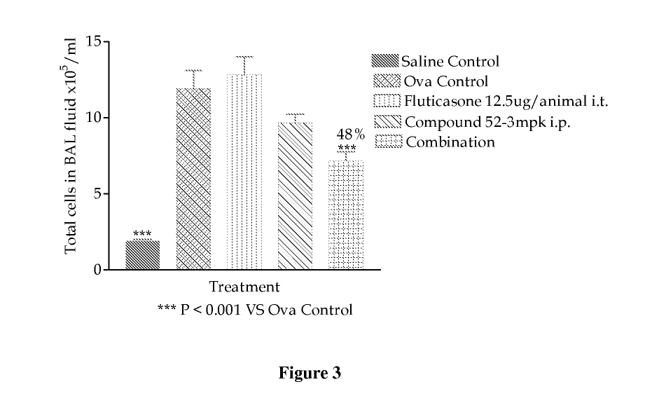

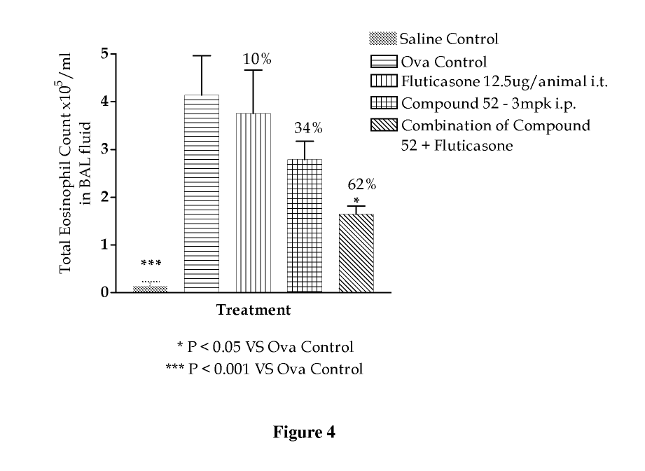

In the ovalbumin challenged-vehicle (Ova Vehicle) treated animals, significant increase in inflammation (eosinophils) was observed compared to saline controls (Saline Vehicle) (FIGS. 3 and 4).

### Conclusion

Compound 52 in combination with fluticasone showed significant synergy in inhibition of eosinophilia in asthma model in Brown Norway rats. The combination of Compound 52 and budesonide showed synergistic effect compared to the respective monotherapy arms.

### Example 4

### Animal Studies for the Combination of TRPA1 Antagonist and Budesonide

Female BALB/c mice (18-20 g on day 0) were sensitized with an i.p. injection of a 0.25-ml suspension containing OVA (50 μg) and aluminum hydroxide (imjet alum, 4 mg) in 0.9% saline. Control animals received 0.25 ml of AHG (4.0 mg/ml) in 0.9% saline. Mice were challenged with OVA aerosol (3%) for 60 minutes in mass dosing chamber using Hudson Nebulizer.

### Animal Grouping

Animals were assigned to one of the following 5 groups during each experiment (Table 5).

A; Vehicle treated/Aluminium Hydroxide Gel Sensitized/Saline challenged
B: Vehicle treated/Ovalbumin sensitized/Ovalbumin challenged
C: Compound 52 treated/Ovalbumin sensitized/Ovalbumin challenged
D: Budesonide treated/Ovalbumin sensitized/Ovalbumin challenged
E: Compound 52+Budesonide treated/Ovalbumin sensitized/Ovalbumin challenged.

TABLE 5

| Group | Group Code | Dose | Ova Challenge |
|---|---|---|---|
| A | Saline Vehicle | 10 ml/kg | − |
| B | Ova Vehicle | 10 ml/kg | + |
| C | Compound 52 | 1 mg/kg | + |
| D | Budesonide | 0.3 mg/kg | + |
| E | Compound 52 + | 1 mg/kg + 0.3 mg/kg | + |
| | Budesonide | | |

### Compound Administration

Sensitized mice were randomly assigned to different treatment groups. Test compounds were triturated and volume was made up with 0.5% CMC. Animals were administered Compound 52 orally from Day-11 to 14. Animals were administered budesonide orally bid from day 11 to day 14.

Approximately 24 hrs post final OVA challenge, animals were euthanized by over dose of Urethane. BAL was performed with (0.3 ml×4 times, EDTA 10 μl) PBS (pH 7.4). Total leukocyte count was done by transferring 20 μl of the BAL fluid in 20 μl Turk Solution. Further, the BAL fluid was centrifuged at 10000 rpm for 10 min at 4° C. Pellet was suspended in 15 μl serum for preparation of smear. For cell differentials, slides were stained with Leishman's stain and a differential cell count of 500 cells based on standard morphology was performed manually.

The total number of eosinophils in each BAL sample was calculated using the formula:

$$Total No . of eosinophils ( in B A L ) = \frac{Total cell count \times {10}^{5} \text{/} mL \times \% eosinophils}{100}$$

Percent inhibition of eosinophils was calculated using the following formula:

$$\% Inhibition of eosinophils = \frac{Avg . {eosinophils}_{( Ova + Veh )} - {eosinophils}_{( treatment )}}{Avg . {eosinophils}_{( Ova + Veh )} - Avg . {eosinophils}_{( Saline + Veh )}} \times 100$$

Data was statistically evaluated by ANOVA followed by Dunnett's multiple comparisons test.

### Results

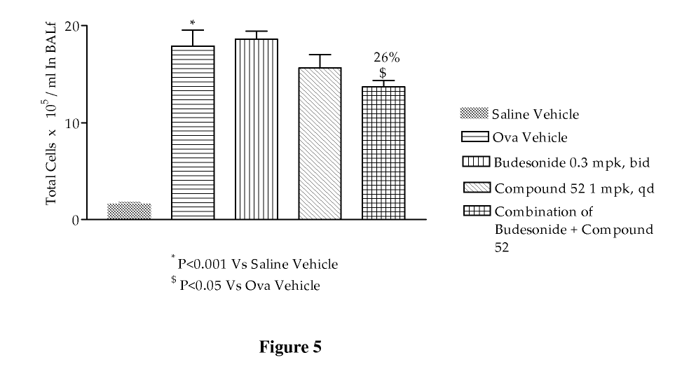

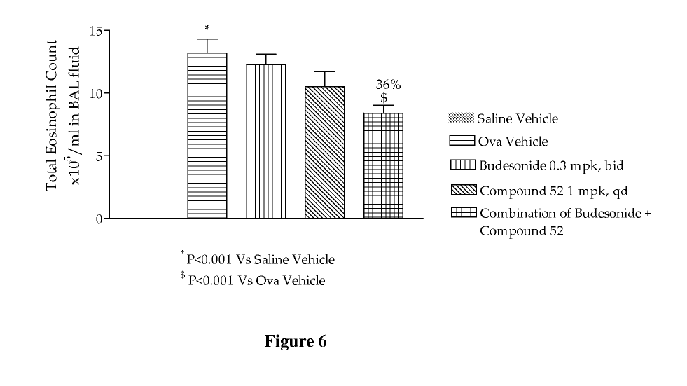

In the ovalbumin challenged-vehicle (Ova Vehicle) treated animals, significant increase in inflammation (total cells and eosinophils) was observed compared to saline controls (Saline Vehicle) (FIG. 3 and FIG. 4). Combination of compound 52 with budesonide showed significant inhibition of total cells and eosinophils (FIG. 5 and FIG. 6).

### Conclusion

Compound 52 in combination with budesonide showed significant synergy in inhibition of eosinophilia in asthma model in mice. The combination of Compound 52 and budesonide showed synergistic effect compared to the respective monotherapy arms.

Although the invention herein has been described with reference to particular embodiments, it is to be understood that these embodiments are merely illustrative of the principles and application of the present invention. It is therefore to be understood that numerous modifications may be made to the illustrative embodiments of the present invention as described.

All publications, patents, and patent applications cited in this application are herein incorporated by reference to the same extent as if each individual publication, patent, or patent application was specifically and individually indicated to be incorporated herein by reference.

1. A pharmaceutical composition comprising synergistically effective amount of a TRPA1 antagonist having an IC₅₀ for inhibiting human TRPA1 receptor activity of less than 1 micromolar, and a glucocorticoid selected from the group consisting of beclomethasone, fluticasone, mometasone, triamcinolone, prednisone, budesonide, ciclesonide, flunisolide or salts thereof.

2. The pharmaceutical composition according to claim 1, wherein the TRPA1 antagonist has an IC₅₀ for inhibiting human TRPA1 receptor activity of less than 500 nanomolar.

3. The pharmaceutical composition according to claim 2, wherein the TRPA1 antagonist has an IC₅₀ for inhibiting human TRPA1 receptor activity of less than 250 nanomolar.

4. The pharmaceutical composition according to claim 1, wherein the TRPA1 antagonist and the glucocorticoid are present in a weight ratio ranging from about 1:0.001 to about 1:5000.

5. (canceled)

6. (canceled)

7. (canceled)

8. A pharmaceutical composition comprising synergistically effective amount of a TRPA1 antagonist having structure of formulae: (XII) or (D) 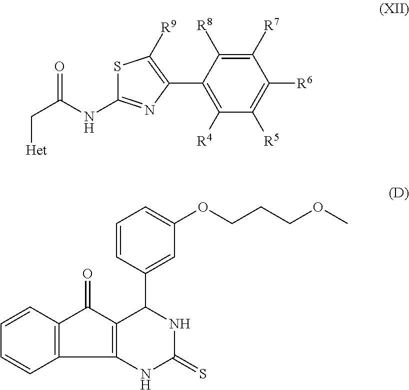
or a pharmaceutically-acceptable salt thereof, wherein,
‘Het’ is selected from the group consisting of 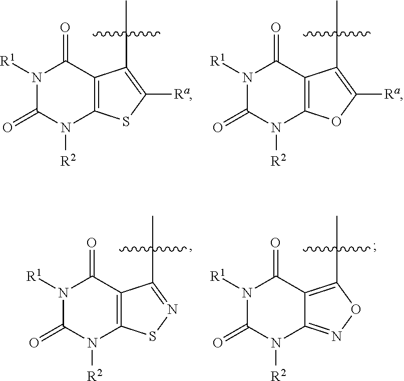
R¹, R² and R^{a}, which may be the same or different, are each independently hydrogen or (C₁-C₄) alkyl;
R⁴, R⁵, R⁶, R⁷, R⁸ and R⁹, which may be same or different, are each independently selected from the group comprising of hydrogen, halogen, cyano, hydroxyl, nitro, amino, substituted or unsubstituted alkyl, alkoxy, haloalkyl, haloalkoxy, cycloalkyl, cycloalkylalkyl, cycloalkenyl, cycloalkylalkoxy, aryl, arylalkyl, biaryl, heteroaryl, heteroarylalkyl, heterocyclic ring and heterocyclylalkyl;
and a glucocorticoid selected from the group consisting of beclomethasone, fluticasone, mometasone, triamcinolone, prednisone, budesonide, ciclesonide, flunisolide or salts thereof.

9. (canceled)

10. (canceled)

11. (canceled)

12. (canceled)

13. (canceled)

14. (canceled)

15. A pharmaceutical composition comprising synergistically effective amount of a TRPA1 antagonist having structure of formula: 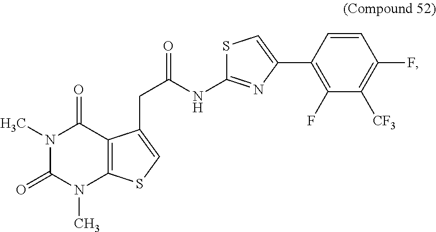 [SMILES: Cn1c(=O)c2c(CC(=O)Nc3nc(-c4ccc(F)c(C(F)(F)F)c4F)cs3)csc2n(C)c1=O]
and a glucocorticoid selected from the group consisting of beclomethasone, fluticasone, mometasone, triamcinolone, prednisone, budesonide, ciclesonide, flunisolide or salts thereof.

16. (canceled)

17. The pharmaceutical composition according to claim 15, wherein the composition is a fixed dose combination.

18. The pharmaceutical composition according to claim 15, wherein the composition is for oral administration, and wherein the TRPA1 antagonist and the glucocorticoid are present in a weight ratio ranging from about 1:0.001 to about 1:100.

19. The pharmaceutical composition according to claim 15, wherein the composition is for inhalation administration, and wherein the TRPA1 antagonist and the glucocorticoid are present in a weight ratio ranging from about 1:0.001 to about 1:5000.

20. (canceled)

21. A method of treating a respiratory disorder in a subject in need thereof, said method comprising administering to the subject the pharmaceutical composition according to claims 15.

22. A method of treating a respiratory disorder by reducing eosinophils count and/or increasing FEV1 value in a subject in need thereof, said method comprising administering to the subject the pharmaceutical composition according to claim 15, thereby reducing said eosinophil count and/or increasing FEV 1 value in said subject.

23. A method of reducing eosinophils count and/or increasing FEV1 value in a subject in need thereof, said method comprising administering to the subject the pharmaceutical composition according to claim 15, thereby reducing said eosinophil count and/or increasing FEV1 value in said subject.

24. (canceled)

25. (canceled)

26. (canceled)

27. (canceled)

28. (canceled)

29. The method according to claim 15, wherein the respiratory disorder is asthma.

30. (canceled)

31. (canceled)

32. (canceled)

33. (canceled)

34. (canceled)

35. (canceled)

36. The method according to any one of claims 15, wherein the respiratory disorder is chronic obstructive pulmonary disease.

37. A pharmaceutical composition comprising synergistically effective amount of a TRPA1 antagonist having structure: 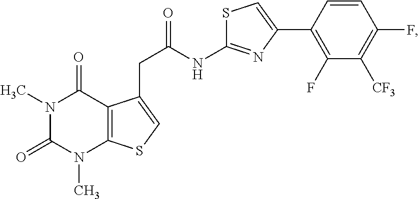 [SMILES: Cn1c(=O)c2c(CC(=O)Nc3nc(-c4ccc(F)c(C(F)(F)F)c4F)cs3)csc2n(C)c1=O]
a glucocorticoid which is fluticasone or its salt and a pharmaceutically acceptable excipient.

38. The pharmaceutical composition according to claim 37, wherein the composition is for oral administration, and wherein the TRPA1 antagonist and the glucocorticoid are present in a weight ratio ranging from about 1:0.001 to about 1:100.

39. The pharmaceutical composition according to claim 37, wherein the composition is for inhalation administration, and wherein the TRPA1 antagonist and the glucocorticoid are present in a weight ratio ranging from about 1:0.001 to about 1:5000.

40. A method of treating a respiratory disorder in a subject in need thereof, said method comprising administering to the subject the pharmaceutical composition according to claim 37.

41. A method of treating a respiratory disorder by reducing eosinophils count and/or increasing FEV1 value in a subject in need thereof, said method comprising administering to the subject the pharmaceutical composition according to claim 37, thereby reducing said eosinophil count and/or increasing FEV1 value in said subject.

42. A method of reducing eosinophils count and/or increasing FEV1 value in a subject in need thereof, said method comprising administering to the subject the pharmaceutical composition according to claim 37, thereby reducing said eosinophil count and/or increasing FEV 1 value in said subject.

43. The method according to claim 37, wherein the respiratory disorder is asthma.

44. The method according to any one of claims 37, wherein the respiratory disorder is chronic obstructive pulmonary disease.

45. A pharmaceutical composition comprising synergistically effective amount of a TRPA1 antagonist having structure: 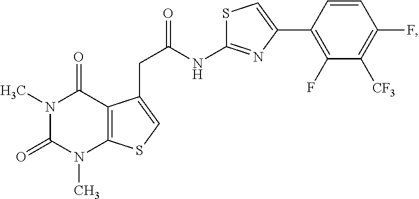 [SMILES: Cn1c(=O)c2c(CC(=O)Nc3nc(-c4ccc(F)c(C(F)(F)F)c4F)cs3)csc2n(C)c1=O]
a glucocorticoid which is budesonide or its salt and a pharmaceutically acceptable excipient.

46. The pharmaceutical composition according to claim 45, wherein the composition is for oral administration, and wherein the TRPA1 antagonist and the glucocorticoid are present in a weight ratio ranging from about 1:0.001 to about 1:100.

47. The pharmaceutical composition according to claim 45, wherein the composition is for inhalation administration, and wherein the TRPA1 antagonist and the glucocorticoid are present in a weight ratio ranging from about 1:0.001 to about 1:5000.

48. A method of treating a respiratory disorder in a subject in need thereof, said method comprising administering to the subject the pharmaceutical composition according to claim 45.

49. A method of treating a respiratory disorder by reducing eosinophils count and/or increasing FEV1 value in a subject in need thereof, said method comprising administering to the subject the pharmaceutical composition according to claim 45, thereby reducing said eosinophil count and/or increasing FEV1 value in said subject.

50. A method of reducing eosinophils count and/or increasing FEV1 value in a subject in need thereof, said method comprising administering to the subject the pharmaceutical composition according to claim 45, thereby reducing said eosinophil count and/or increasing FEV 1 value in said subject.

51. The method according to claim 45, wherein the respiratory disorder is asthma.

52. The method according to any one of claims 45, wherein the respiratory disorder is chronic obstructive pulmonary disease.
